(19) United States
(12) Patent Application Publication
(10) Pub. No.: US 2025/0391069 A1
(43) Pub. Date: Dec. 25, 2025

(54) HEALTH MANAGING SYSTEM AND HEALTH MANAGING METHOD

(71) Applicant: TOTO LTD., Kitakyushu-shi, Fukuoka (JP)

(72) Inventors: Kenta Suzuki, Fukuoka (JP); Tetsuya Uchida, Fukuoka (JP); Teruyuki Kamori, Fukuoka (JP); Ryota Takahashi, Fukuoka (JP); Kaori Kuwahara, Fukuoka (JP)

(21) Appl. No.: 18/880,306

(22) PCT Filed: Jul. 27, 2023
(86) PCT No.: PCT/JP2023/027629

§ 371 Date: Dec. 31, 2024

(30) Foreign Application Priority Data
Jul. 29, 2022 (JP) 2022-122089
Jul. 25, 2023 (JP) 2023-120413

(51) Int. Cl. G06T 11/20; A61B 10/00; G16H 40/67; G16H 50/30

(52) U.S. Cl. CPC G06T 11/206; A61B 10/0038; G16H 40/67; G16H 50/30; G06T 2210/41

(57) ABSTRACT

A health managing system according to an embodiment includes: an excreted stool information acquiring unit that acquires excreted stool information corresponding to a user; an excreted stool state determining unit that determines an excreted stool property of an excreted stool corresponding to the excreted stool information based on the excreted stool information acquired by the excreted stool information acquiring unit; a display to be browsed by the user; and a display processing unit that executes a process for causing the display to display, as time series data, the excreted stool property that is determined by the excreted stool state determining unit. The display processing unit is further configured to: being capable of switching displaying for each predetermined time interval; and from among excreted stool property patterns during the predetermined time interval, execute a process for causing the display to display a predetermined number of ones whose frequency is large.

### FIELD

Embodiment of the disclosure relates to a health managing system and a health managing method.

### BACKGROUND

Conventionally, there has been disclosed a system configured to display a property of a stool (simply referred to as “stool”) excreted by a user in a toilet in which the user is sitting on a toilet seat thereof, so as to visualize states of excreted stools in time series (for example, see Patent Literature 1).

### CITATION LIST

### Patent Literature

Patent Literature 1: Japanese Laid-Open Patent Publication No. 2021-051449

### SUMMARY

### Technical Problem

However, the above-mentioned conventional technology has a room for improvement. For example, the above-mentioned conventional technology merely counts and displays a stool of a user for each type (for example, shape), and the convenience of the information provision is hardly high in accordance with the user, so that the above-mentioned conventional technology has a room for improvement in a point of information provision related to a stool of a user.

It is an object of one aspect of a disclosed embodiment to provide a health managing system and a health managing method capable of appropriate information provision related to a stool of a user.

### Solution to Problem

A health managing system according to one aspect of an embodiment includes: an excreted stool information acquiring unit that acquires excreted stool information corresponding to an excretion of a stool of a user; an excreted stool state determining unit that determines an excreted stool property of an excreted stool corresponding to the excreted stool information based on the excreted stool information acquired by the excreted stool information acquiring unit; a display to be browsed by the user; and a display processing unit that executes a process for causing the display to display, as time series data, the excreted stool property that is determined by the excreted stool state determining unit, wherein the display processing unit is further configured to: be capable of switching displaying for each predetermined time interval; and from among excreted stool property patterns during the predetermined time interval, execute a process for causing the display to display a predetermined number of ones whose frequency is large.

In accordance with a health managing system according to one aspect of an embodiment, a tendency is not grasped if all samples are displayed at displaying corresponding to a predetermined time interval, and thus a representative value is described, so that it is possible to easily grasp a tendency, and further to report precise health information. As described above, the health managing system enables appropriate information provision related to a stool of a user. For example, in a case where a stool property, particularly a color, a type (for example, shape), and the like are displayed for each time interval by using, for example, an average value thereof; a stool property deviates (in other words, is changed) from a quantification result in some cases. Generally, even if excreted stool states are enumerated in grasping a daily tendency, a user hardly understand which state is an ideal one. In a case of time series enumeration, an information amount is large, and thus a user hardly grasps a tendency in excreted stool at a glance. However, according to the health managing system, it is possible to display information in an appropriate mode in which displaying can be switched from among predetermined time intervals, and from among excreted stool property patterns during the predetermined time interval, a predetermined number of ones whose frequency is large are displayed.

In the health managing system according to one aspect of the embodiment, the display processing unit is further configured to: execute a process for switching the displaying on the display from among a day unit, a week unit, a month unit, and a year unit.

In accordance with the health managing system according to one aspect of the embodiment, a user is able to grasp a tendency at a timing (namely, time interval) wanted by him/her. Therefore, the health managing system enables appropriate information provision related to a stool of a user.

In the health managing system according to one aspect of the embodiment, the display processing unit is further configured to: in a case where executing displaying by at least a month unit or a year unit, execute a process for causing the display to display a predetermined number of patterns whose frequency is large from among patterns corresponding to excreted stool properties during the predetermined time interval.

In accordance with the health managing system according to one aspect of the embodiment, even in a case where a lot of data corresponding to a week, a month, a year, and the like; a user is able to easily grasp a tendency. Therefore, the health managing system enables appropriate information provision related to a stool of a user.

In the health managing system according to one aspect of the embodiment, the display processing unit is further configured to: in a case where executing displaying by at least a day unit, display all excreted stool properties of the user determined by the excreted stool state determining unit.

In accordance with the health managing system according to one aspect of the embodiment, all data are displayed in a case of a day unit, a user is able to grasp a precise tendency. Therefore, the health managing system enables appropriate information provision related to a stool of a user.

In the health managing system according to one aspect of the embodiment, the excreted stool state determining unit is further configured to: determine the excreted stool property including a type, an amount, and a color of an excreted stool, and the display processing unit is further configured to: execute a process for causing the display to execute displaying based on a pattern corresponding to a combination of the type, the amount, and the color.

In accordance with the health managing system according to one aspect of the embodiment, important three features of a stool are set to be items, and thus a user is able to grasp a precise tendency. Therefore, the health managing system enables appropriate information provision related to a stool of a user.

In the health managing system according to one aspect of the embodiment, the display processing unit is further configured to: execute a process for causing the display to display the pattern based on a preference order of the type, the amount, and the color in this order.

In accordance with the health managing system according to one aspect of the embodiment, a health state is displayed at a higher rank while prioritizing a better state. For example, the health managing system ranks a type of a stool in preference to a color of the stool (for example, ranks type 4 in preference to yellow color), so that it is possible to associate information with a preference order so as to present it to a user. Therefore, the health managing system enables appropriate information provision related to a stool of a user.

A health managing method according to one aspect of the embodiment includes: acquiring excreted stool information corresponding to an excretion of a stool of a user; determining an excreted stool property of an excreted stool corresponding to the excreted stool information based on the excreted stool information acquired in the acquiring; and executing a process for causing a display to be browsed by the user to display, as time series data, the excreted stool property that is determined in the determining, wherein the executing includes: being capable of switching displaying for each predetermined time interval; and from among excreted stool property patterns during the predetermined time interval, execute a process for causing the display to display a predetermined number of ones whose frequency is large.

In accordance with a health managing method according to one aspect of an embodiment, a tendency is not grasped if all samples are displayed at displaying corresponding to a predetermined time interval, and thus a representative value is described, so that it is possible to easily grasp a tendency, and further to report precise health information. As described above, the health managing system enables appropriate information provision related to a stool of a user. For example, in a case where a stool property, particularly a color, a type (for example, shape), and the like is displayed for each time interval by using, for example, an average value thereof; a stool property deviates (in other words, is changed) from a quantification result in some cases. Generally, even if excreted stool states are enumerated in grasping a daily tendency, a user hardly understand which state is an ideal one. In a case of time series enumeration, an information amount is large, and thus a user hardly grasps a tendency in excreted stool at a glance. However, according to the health managing method, it is possible to display information in an appropriate mode in which displaying can be switched from among predetermined time intervals, and from among excreted stool property patterns during the predetermined time interval, a predetermined number of ones whose frequency is large are displayed.

A health managing system according to one aspect of the embodiment includes: an excreted stool information acquiring unit that acquires excreted stool information corresponding to an excretion of a stool of a user; an excreted stool state determining unit that determines an excreted stool property of an excreted stool corresponding to the excreted stool information based on the excreted stool information acquired by the excreted stool information acquiring unit; a display to be browsed by the user; and a display processing unit that executes a process for causing the display to display, as time series data, the excreted stool property that is determined by the excreted stool state determining unit, wherein the excreted stool state determining unit is further configured to: determine a type of a stool, an amount of the stool, and a color of the stool, and the display processing unit is further configured to execute a process including: executing time series displaying on stool by using a graph in which a lateral axis corresponds to time and a vertical axis corresponds to a pattern of a stool such that a color of each point in the graph indicates a color of a stool, and a size of the corresponding point indicates an amount of the stool.

In accordance with the health managing system according to one aspect of the embodiment, time series displaying of a stool is executed by using a graph in which a lateral axis corresponds to time and a vertical axis corresponds to a pattern of a stool, the displaying is executed such that a color of each point in the graph indicates a color of a stool and a size of the corresponding point indicates an amount of the stool, and time series displaying is executed by a mode, such as a color and a size of a point, that allows a user intuitive recognition, so that it is possible to easily grasp a tendency of an excreted stool along a time series, and further to report precise health information. As described above, the health managing system enables appropriate information provision related to a stool of a user.

### Advantageous Effects of Invention

According to one aspect of an embodiment, it is possible to achieve appropriate information provision related to a stool of a user.

### BRIEF DESCRIPTION OF DRAWINGS

FIG. 1 is a schematic perspective view illustrating a toilet system according to an embodiment.

FIG. 2 is a schematic side view illustrating the toilet system according to the embodiment.

FIG. 3 is a diagram illustrating a configuration example of a health managing system according to the embodiment.

FIG. 4 is a schematic diagram illustrating various health index calculating processes based on a quantification result of a biological sensor.

FIG. 5 is a diagram illustrating one example of a calculation method of a numeric value.

FIG. 6 is a diagram illustrating one example of a calculation method of a numeric value.

FIG. 7 is a diagram illustrating one example of a calculation method of a numeric value.

FIG. 8 is a flowchart illustrating one example of a procedure for processes to be executed by the health managing system.

FIG. 9 is a diagram illustrating a displaying example of a health score.

FIG. 10 is a diagram illustrating a displaying example of information associated with health.

FIG. 11 is a diagram illustrating a display example according to a time interval.

FIG. 12 is a diagram illustrating one example of a preference order of a stool property.

FIG. 13 is a diagram illustrating one example of time series displaying of stools.

FIG. 14 is a diagram illustrating one example of an explanation displaying of a stool property.

FIG. 15 is a diagram illustrating one example of time series displaying of health indexes.

FIG. 16 is a diagram illustrating one example of time series displaying of health indexes.

### DESCRIPTION OF EMBODIMENTS

Hereinafter, an embodiment of a health managing system and a health managing method according to the present application will be described in detail with reference to the accompanying drawings. The present disclosure is not limited to the embodiment described in the following.

### <1. Configuration of Health Managing System>

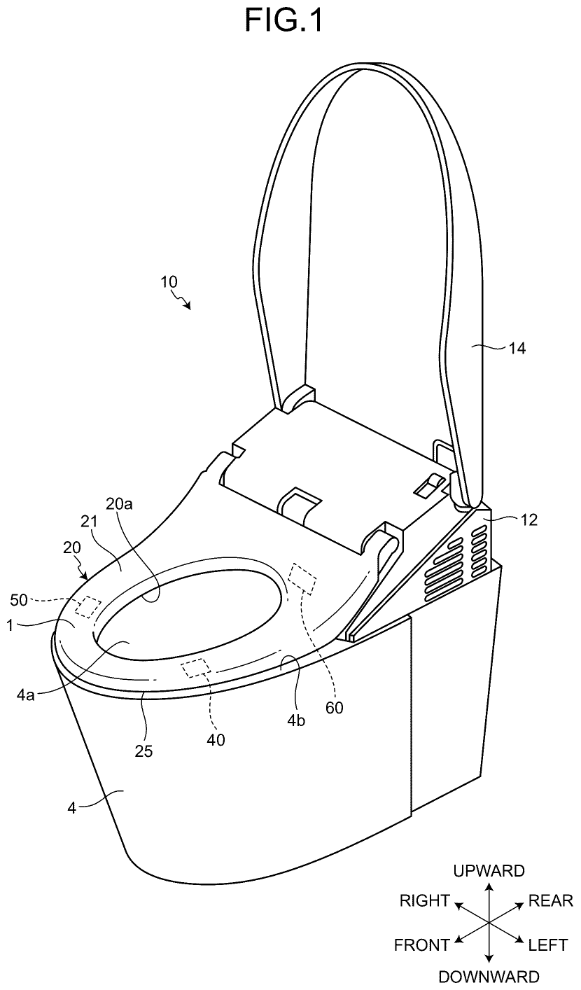

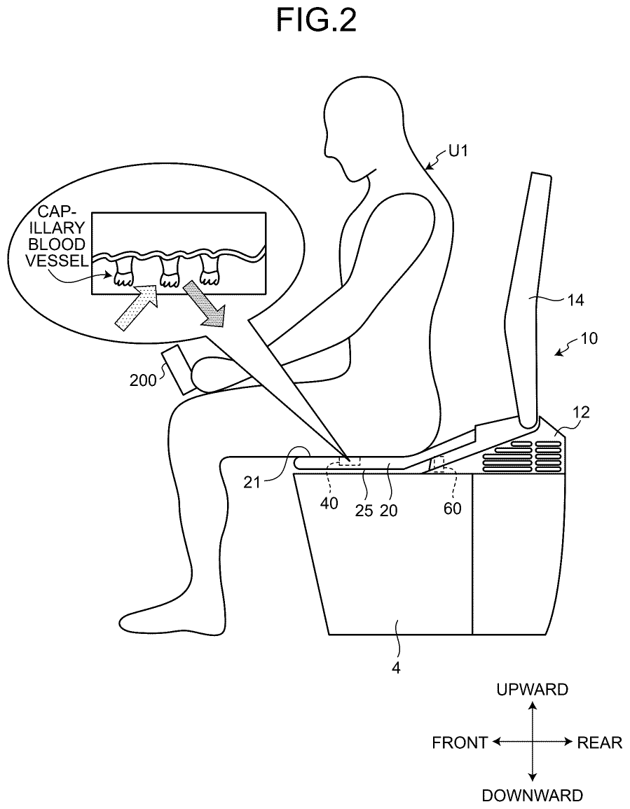

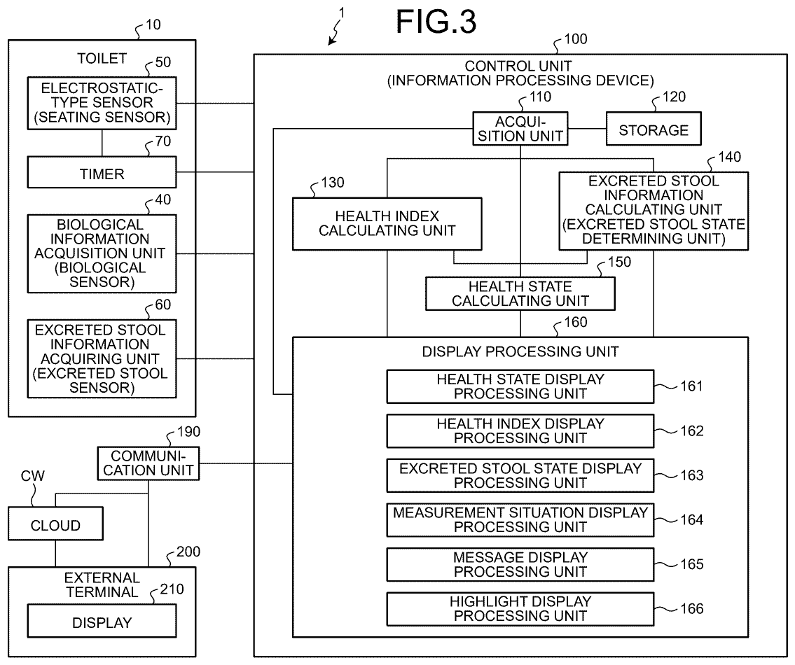

A configuration of a health managing system according to the embodiment will be explained with reference to FIG. 1 to FIG. 3. FIG. 1 is a schematic perspective view illustrating a toilet system according to the embodiment. FIG. 2 is a schematic side view illustrating the toilet system according to the embodiment. FIG. 3 is a diagram illustrating a configuration example of the health managing system according to the embodiment.

A health managing system 1 according to the embodiment to be mentioned hereinafter is configured to cause a sensor to measure (may be also referred to as “quantify”) various kinds of information related to a user (namely, user), and further to calculate a health score on the basis of the measured (namely, quantified) information. Note that processes to be explained hereafter whose processing subject is the health managing system 1 may be any device(s) capable of executing the processes in accordance with a device configuration included in the health managing system 1.

As illustrated in FIG. 3, the health managing system 1 includes a toilet 10 including a biological sensor 40, a seating sensor 50, and an excreted stool sensor 60; a control unit 100; and an external terminal 200. The health managing system 1 may include the plurality of toilets 10, the plurality of control units 100, and/or the plurality of external terminals 200. Note that the device configuration illustrated in FIG. 3 is merely one example, and the health managing system 1 may include a server device that is configured to provide, to the control unit 100, information to be used by the control unit 100 calculating various kinds of information such as a health score, for example.

A configuration of the toilet 10 will be explained in addition to the outline of an exterior (namely, physical) configuration of the toilet 10 that is one example of the toilet system. As illustrated in FIG. 1 and FIG. 2, the toilet 10 includes a toilet seat 20, a main body part 12, and a toilet lid 14. Each of the toilet seat 20 and the toilet lid 14 is supported by the main body part 12 to be rotatable.

As illustrated in FIG. 3, the toilet 10 includes the biological sensor 40, the seating sensor 50, the excreted stool sensor 60, and a timer 70. Details thereof will be mentioned later, the biological sensor 40 is a sensor for detecting (namely, acquiring) biological information on a user, the seating sensor 50 is a sensor for detecting sitting of a user, and the excreted stool sensor 60 is a sensor for detecting an excreted stool (may be also referred to as “stool”) of a user. For example, the biological sensor 40 is a laser sensor configured to quantify a physical amount reflecting blood flow information of a user, and the excreted stool sensor 60 is an image capturing device (namely, image sensor) configured to capture an image of an excreted stool of a user. The biological sensor 40 and the seating sensor 50 are provided to the toilet seat 20, and the excreted stool sensor 60 is provided to the main body part 12. Note that details of the biological sensor 40, the seating sensor 50, and the excreted stool sensor 60 will be mentioned later.

As illustrated in FIG. 1, the toilet seat 20 includes an opening 20a. The O-shaped opening 20a is formed in a center portion of the toilet seat 20. The opening of the toilet seat 20 is not limitedly formed in O-shaped, and may be formed in U-shaped or the like. The periphery of the toilet seat 20 is formed so as to curve along an outer shape of a closet bowl 4. The toilet seat 20 is commonly formed by an opaque resin material (for example, polypropylene) so as to include a seating surface 21 on which a user sits and a bottom surface 25 on an opposite side of the seating surface 21.

The seating surface 21 is a surface that is exposed upward in a state where the toilet seat 20 is placed on an upper surface 4b of the closet bowl 4 so as to be a surface on which a user sits. The bottom surface 25 is a surface that faces the upper surface 4b of the closet bowl 4 in a state where the toilet seat 20 is put down. Almost whole of the toilet seat 20 is thickly formed, and a part (may be referred to as “thin portion”) thinner than other part is locally formed in a position corresponding to the biological sensor 40.

Note that the toilet seat 20 may be provided therein with a heater wire and thermal insulation that warms the seating surface 21 and keeps the seating surface 21 warm, and the like. The heater wire is controlled by a toilet seat heating unit provided in the main body part 12, and is extended throughout an inner part of the toilet seat 20 so as not to interfere with the biological sensor 40, the seating sensor 50, and the excreted stool sensor 60. The thermal insulation is arranged under the heater wire, the biological sensor 40, and the seating sensor 50.

A part (namely, thin portion) corresponding to the biological sensor 40 of the toilet seat 20 has a thickness through which irradiation light irradiated from the biological sensor 40 and reflection light reflected from a user sitting on the seating surface 21 are able to pass. The thickness of the thin portion is set depending on the intensity of the irradiation light from the biological sensor 40 and the reflection light, durability of the toilet seat 20, and the like, so as to be approximately 0.5 mm to 1.0 mm, for example.

The following “upward”, “downward”, “front”, “rear”, “left”, and “right” indicate directions that are viewed from a user sitting on the toilet seat 20 while directing his/her back to the opened toilet lid 14.

The thin portion is formed on the left side in a position in front side from the center of a length in a front-back direction of the opening 20a of the toilet seat 20 so as to locate in a position in front and left side from a gravity center position of the user sitting on the toilet seat 20. Thus, as illustrated in FIG. 2, the thin portion faces (namely, is in contact with) a reverse side of a left thigh of a user (for example, user U1 illustrated in FIG. 2) sitting on the toilet seat 20. In FIG. 2, a state is illustrated where the user U1 grips the external terminal 200 with his/her left hand.

The thin portion is formed as small as possible within a range where the biological sensor 40 can detect blood flow information on a user sitting on the toilet seat 20, and is formed in round-shaped whose diameter is equal to or less than 12 mm (preferably, equal to or less than 8 mm), for example.

The main body part 12 is arranged behind a bowl part of the closet bowl 4 to be attached to the upper surface 4b of the closet bowl 4. The main body part 12 is provided therein with an opening/closing unit configured to control an opening/closing operation of the toilet seat 20 and the toilet lid 14, a toilet seat heating unit configured to control the temperature of the toilet seat 20, a washing unit configured to wash a private part of a human body, and a deodorizing unit configured to reduce odor components. For example, units 12a to 12d are controlled by the control unit 100. For example, the excreted stool sensor 60 is arranged in a position facing a bowl part of the closet bowl 4 from the main body part 12, and further not interfering with the units 12a to 12d.

The biological sensor 40 functions as a biological information acquiring unit that acquires biological information. For example, the biological sensor 40 includes a laser sensor so as to acquire blood flow information on a user as biological information on the user. As illustrated in FIG. 1 and FIG. 2, the biological sensor 40 is arranged on a reverse face of the thin portion in the toilet seat 20. The biological sensor 40 is a reflection-type sensor that is configured to irradiate infrared irradiation light toward a reverse side of a left thigh in a user so as to detect reflection light (scattered light generating Doppler shift caused by red blood cells) in accordance with a blood flow state of blood vessels under the skin.

For example, the biological sensor 40 is a laser sensor that is capable of quantifying a blood flow state in the skin on the basis of a Dynamic Light Scattering method (Dynamic Light Scattering). In FIG. 2, the biological sensor 40 is attached to a portion of the toilet seat 20, which is corresponding to a reverse side of a thigh in a user.

Note that the blood flow information is merely one example, an arbitrary sensor may be employed for the biological sensor 40 in accordance with biological information to be acquired. For example, in a case where acquiring information related to heartbeat (pulse wave) of a user as biological information on the user, the biological sensor 40 may be a heartbeat sensor. In a case where acquiring information related to respiration of a user as biological information on the user, the biological sensor 40 may be a respiration sensor. In a case where acquiring information related to pulse (pulse wave) of a user as biological information on the user, the biological sensor 40 may be a pulse sensor. In a case where acquiring information related to heart movement of a user as biological information on the user, the biological sensor 40 may be an electrocardiograph.

The biological sensor 40 is connected to the control unit 100 via a predetermined network to be able to communicate with each other in a wired or wireless manner. The biological sensor 40 transmits various kinds of information to the control unit 100. For example, the biological sensor 40 transmits acquired biological information on a user to the control unit 100. For example, the biological sensor 40 may be connected to the control unit 100 to be able to communicate with each other by a predetermined wireless communication function such as Bluetooth (Registered Trademark) and Wireless Fidelity (Wi-Fi: Registered Trademark). Note that the control unit 100 and the biological sensor 40 may be connected to each other in any connection as long as information is transmitted and received therebetween, and they may be connected to be able to communicate with each other in a wired manner, or they may be connected to be able to communicate with each other in a wireless manner. For example, the biological sensor 40 may be connected to the control unit 100 via a communication unit 190 to be able to communicate with each other in a wired or wireless manner.

The seating sensor 50 includes a function for detecting sitting of a person on the toilet seat 20. The seating sensor 50 detects that a user seats him/herself (namely, sits) on the toilet seat 20. The seating sensor 50 is capable of detecting a user sitting on the toilet seat 20. The seating sensor 50 also functions as an unseating detection sensor configured to detect a user unseating from the toilet seat 20. The seating sensor 50 detects a seated state of a user with respect to the toilet seat 20.

For example, the seating sensor 50 includes an electrostatic-type sensor. As illustrated in FIG. 1 and FIG. 2, the seating sensor 50 is formed on the right side in a position in front side from the center of a length in a front-back direction of the opening 20a of the toilet seat 20 so as to locate in a position in front and right side from a gravity center position of a user sitting on the toilet seat 20. Thus, in a case where facing (in other words, being in contact with) a reverse side of a right thigh of a user sitting on the toilet seat 20, the seating sensor 50 is capable of detecting the above-mentioned seated state.

Note that the case where the seating sensor 50 includes an electrostatic-type sensor is merely one example, and the seating sensor 50 may have any detection type, and further may be arranged in any position as long as being capable of detecting sitting of a user on the toilet seat 20. For example, in a case where the seating sensor 50 is a distance sensor having an infrared ray type or a micro (μ) wave type so as to detect sitting on the basis of a distance, the seating sensor 50 may be arranged in a position for detecting a leg of a person from a side of the closet bowl 4 or may be arranged in a position for detecting a back of a person from a tank attached to the closet bowl 4. For example, in a case where detecting sitting on the basis of a distance, the seating sensor 50 may be arranged in a ceiling of a space (namely, toilet room) in which the closet bowl 4 is provided. For example, in a case where the seating sensor 50 is a contact switch so as to detect ups and downs of a toilet seat by sitting, the seating sensor 50 may be arranged in a part that supports the toilet seat 20 to be rotatable. For example, in a case where the seating sensor 50 is a load sensor so as to detect sitting on the basis of a weight applied to a toilet seat, the seating sensor 50 may be arranged on a surface of a reverse face of the toilet seat 20, which is in contact with the closet bowl 4.

The seating sensor 50 is connected to the control unit 100 via a predetermined network to be able to communicate with each other in a wired or wireless manner. The seating sensor 50 transmits various kinds of information to the control unit 100. For example, the seating sensor 50 transmits acquired information related to sitting (or unseating) of a user to the control unit 100. For example, the seating sensor 50 may be connected to the control unit 100 to be able to communicate with each other by a predetermined wireless communication function such as Bluetooth and Wi-Fi. Note that the control unit 100 and the seating sensor 50 may be connected to each other in any connection as long as information is transmitted and received therebetween, and they may be connected to be able to communicate with each other in a wired manner, or they may be connected to be able to communicate with each other in a wireless manner. For example, the seating sensor 50 may be connected to the control unit 100 via the communication unit 190 to be able to communicate with each other in a wired or wireless manner.

The excreted stool sensor 60 functions as an excreted stool information acquiring unit configured to acquire excreted stool information corresponding to an excreted stool of a user. For example, the excreted stool sensor 60 includes an image sensor that captures inside of the closet bowl 4 so as to acquire excreted stool information on an excreted stool corresponding to a user. For example, the excreted stool sensor 60 is an image sensor that is arranged in a position facing a bowl part of the closet bowl 4 from the main body part 12. For example, an arbitrary sensor such as a Charge Coupled Device (CCD) sensor and a Complementary Metal Oxide Semiconductor (CMOS) sensor is used for the excreted stool sensor 60.

For example, the excreted stool sensor 60 may be a line sensor (one-dimensional image sensor) that captures a one-dimensional image, or may be an area sensor (two-dimensional image sensor) that captures a two-dimensional image. For example, in a case of a line sensor, the excreted stool sensor 60 is arranged in a direction for capturing a space between the toilet seat 20 and a water sealing part (namely, part retaining therein sealing water) of the closet bowl 4. As described above, the excreted stool sensor 60 may be arranged so as to capture a falling stool until the stool reaches water retained in the water sealing part (namely, part retaining therein sealing water) of the closet bowl 4 after excretion of a user.

For example, in a case of an area sensor, the excreted stool sensor 60 is arranged in a direction for capturing the water sealing part of the closet bowl 4. As described above, the excreted stool sensor 60 may be arranged so as to capture the water sealing part of the closet bowl 4. Note that the excreted stool sensor 60 may have any arrangement mode as long as being capable of detecting (namely, capturing) a stool. The excreted stool sensor 60 may capture a still image, or may capture a video image. In a case where inside of the closet bowl becomes dark when a user sits on a toilet seat so as not to be capable of capturing an image with a sufficient brightness, a light source (namely, light emitting unit) may be provided.

The excreted stool sensor 60 is connected to the control unit 100 via a predetermined network to be able to communicate with each other in a wired or wireless manner.

The excreted stool sensor 60 transmits various kinds of information to the control unit 100. For example, the excreted stool sensor 60 transmits, to the control unit 100, acquired information related to an excreted stool of a user. For example, the excreted stool sensor 60 may be connected to the control unit 100 to be able to communicate with each other by a predetermined wireless communication function such as Bluetooth and Wi-Fi. Note that the control unit 100 and the excreted stool sensor 60 may be connected to each other in any connection as long as information is transmitted and received therebetween, and they may be connected to be able to communicate with each other in a wired manner, or they may be connected to be able to communicate with each other in a wireless manner. For example, the excreted stool sensor 60 may be connected to the control unit 100 via the communication unit 190 to be able to communicate with each other in a wired or wireless manner.

In a case where explaining information acquired by the biological sensor 40 and information acquired by the excreted stool sensor 60 without differentiating therebetween, they may be comprehensively referred to as “sensor information”. The sensor information is a concept including information that is acquired by various sensors in order to estimate a state related to health of a user, such as information acquired by the biological sensor 40 and information acquired by the excreted stool sensor 60.

The timer 70 measures time. The timer 70 quantifies a time interval during which a sensor is acquiring sensor information. The timer 70 measures a time point at which the seating sensor 50 detects sitting of a user. The timer 70 measures a time interval from a time point when sitting of a user is started, which is detected by the seating sensor 50. The timer 70 may measure a time interval from a time point when the biological sensor 40 starts to acquire biological information. The timer 70 may measure a time point when the excreted stool sensor 60 first detects an acquired stool. The timer 70 may acquire a time interval needed for excretion, which is a difference between a time point when the seating sensor 50 detects sitting of a user and a time point when the excreted stool sensor 60 first detects an acquired stool.

The timer 70 is connected to the control unit 100 via a predetermined network to be able to communicate with each other in a wired or wireless manner. The timer 70 transmits various kinds of information to the control unit 100. For example, the timer 70 transmits information related to measured time to the control unit 100. For example, the timer 70 may be connected to the control unit 100 to be able to communicate with each other by a predetermined wireless communication function such as Bluetooth and Wi-Fi. Note that the control unit 100 and the timer 70 may be connected to each other in any connection as long as information is transmitted and received therebetween, and they may be connected to be able to communicate with each other in a wired manner, or they may be connected to be able to communicate with each other in a wireless manner. For example, the timer 70 may be connected to the control unit 100 via the communication unit 190 to be able to communicate with each other in a wired or wireless manner.

The toilet 10 may include a personal identification unit (namely, identification device) configured to execute a process (namely, personal identification) for specifying a user that is using the closet bowl 4. For example, the personal identification unit of the toilet 10 acquires information for specifying a user using the closet bowl 4 for excretion on the basis of communication with the external terminal 200 owned by the user, an operation of the user with respect to a remote controller, and the like, so as to execute personal identification of the user. For example, the personal identification unit of the toilet 10 communicates with the external terminal 200 owned by a user so as to receive, from the external terminal 200, user identifying information for specifying the user. For example, the personal identification unit of the toilet 10 may receive, from a remote controller, operation information indicating an operation of a user. Note that the personal identification unit of the toilet 10 may specify a user by any method as long as being capable of specifying a user that excretes by using the closet bowl 4.

The personal identification unit of the toilet 10 is connected to the control unit 100 via a predetermined network to be able to communicate with each other in a wired or wireless manner. The personal identification unit of the toilet 10 transmits various kinds of information to the control unit 100. For example, the personal identification unit of the toilet 10 transmits acquired user identifying information on a user to the control unit 100. For example, the personal identification unit of the toilet 10 may be connected to the control unit 100 to be able to communicate with each other by a predetermined wireless communication function such as Bluetooth and Wi-Fi. Note that the control unit 100 and the personal identification unit of the toilet 10 may be connected to each other in any connection as long as information is transmitted and received therebetween, and they may be connected to be able to communicate with each other in a wired manner, or they may be connected to be able to communicate with each other in a wireless manner. For example, the personal identification unit of the toilet 10 may be connected to the control unit 100 via the communication unit 190 to be able to communicate with each other in a wired or wireless manner.

The control unit 100 is an information processing device (namely, computer) configured to execute various kinds of information processing. The control unit 100 is connected to the external terminal 200 via a predetermined network (e.g., communication unit 190) such as the Internet to be able to communicate with each other in a wired or wireless manner. Note that the control unit 100 and the external terminal 200 may be connected to each other in any connection as long as information is transmitted and received therebetween, and they may be connected to be able to communicate with each other in a wired manner, or they may be connected to be able to communicate with each other in a wireless manner. Note that as described above, the control unit 100 is capable of transmitting and receiving information to and from configuration elements of the toilet 10.

The control unit 100 may be arranged in an

arbitrary part. The control unit 100 may be arranged in a space (namely, toilet room) corresponding to the toilet 10 or may be arranged outside of the space (namely, toilet room) corresponding to the toilet 10. The control unit 100 may be arranged in the main body part 12. For example, the control unit 100 may be arranged in a position (position comparatively near biological sensor 40) near a front end of the toilet seat 20. In this case, the control unit 100 is capable of processing an output signal from the biological sensor 40 so as to convert it into a signal having a comparatively strong noise resistance.

Note that regarding a device configuration and arrangement of the control unit 100, an arbitrary mode may be employed as long as the control unit 100 communicates with the external terminal 200 and configuration elements of the toilet 10 to be capable of realizing the processing. For example, the control unit 100 may be a portable terminal (namely, device) such as a laptop computer that can be carried by a management person of the health managing system 1 and the like. Moreover, the control unit 100 may be arranged in the toilet 10. The control unit 100 may be provided (namely, constructed) not in the main body part 12, but in the external terminal 200 with which the control unit 100 communicates via the communication unit 190, and/or in an external network (for example, in cloud CL).

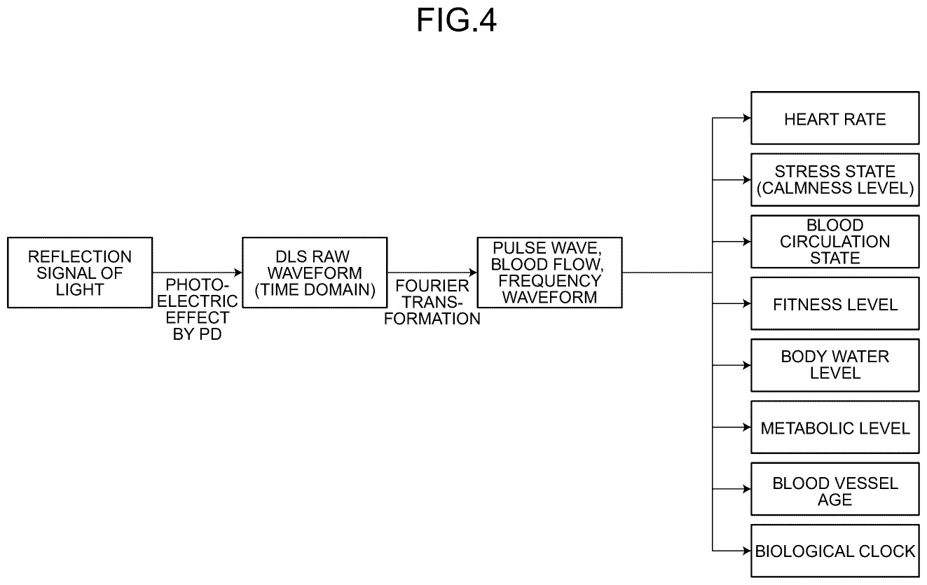

The control unit 100 calculates a health index of a user sitting on the toilet seat 20 on the basis of quantification result of the biological sensor 40. The control unit 100 calculates a health index of a user on the basis of biological information on the user, which is acquired by the biological sensor 40. FIG. 4 is a schematic diagram illustrating various health index calculating processes based on a quantification result of the biological sensor. The control unit 100 causes the biological sensor 40, which is a laser sensor capable of detecting blood flow, to quantify a pulse rate, a pulse fluctuation, a blood flow volume, and the like. On the basis of the quantification result of the biological sensor 40, the control unit 100 calculates a plurality of health indexes such as a heart rate, a stress state (e.g., calmness level), a blood circulation state, a fitness level, a body water level, a metabolic level, a blood vessel age, and a biological clock.

For example, a heart rate is a health index indicating the number of heat-beats of a user during a predetermined time interval. For example, a stress state (may be also referred to as “relax level”) is a health index indicating a state related to the quantity of stress in a user. For example, a blood circulation state is a health index indicating a state related to blood circulation of a user. For example, the blood circulation state may be a blood circulation state (namely, lower limb blood flow state) in a lower limb of a user. Note that the blood circulation state is not limited to that in a lower limb, but may be a blood circulation state in an arbitrary part of a user. For example, the fitness level is a health index related to an aerobic exercise capacity of a user, and the like. The body water level is a health index indicating a ratio (namely, body water content) of a body water amount to a body weight of a user. For example, the metabolic level is a health index related to metabolism of a user. The blood vessel age is a health index related to a blood vessel age of a user. The biological clock is a health index related to a biological clock of a user.

The control unit 100 determines an excreted stool property (may be also referred to “stool property”) of a user sitting on the toilet seat 20 on the basis of a quantification result of the excreted stool sensor 60. The excreted stool property (namely, stool property) means information that indicates a state of an excreted stool (namely, stool) of a user. For example, the excreted stool property (may be also referred to “excreted stool state” or “stool state”) includes a type, a color, and an amount of a stool, etc. The control unit 100 determines a stool state of a user on the basis of excreted stool information on the user which is acquired by the excreted stool sensor 60. For example, the control unit 100 determines a stool state including a type, a color, and an amount of a stool, etc., on the basis of a quantification result of the excreted stool sensor 60 that captures an image. On the basis of a stool state, the control unit 100 calculates a score (namely, excreted stool score) of a stool corresponding to the stool state.

On the basis of a quantification result of the timer 70, the control unit 100 measures a time interval during which a user is sitting on the toilet seat 20. The control unit 100 determines whether or not a time interval during which a user is sitting on the toilet seat 20 reaches a time interval (may be also referred to “necessary time interval”) needed for acquisition (namely, measurement) of information. The control unit 100 determines whether or not a time interval during which a user is sitting on the toilet seat 20 reaches a first time interval. The control unit 100 determines whether or not a time interval during which a user is sitting on the toilet seat 20 reaches a second time interval.

### <1-1. Indexes of Health Index, Stool State, and the Like>

As described above, the control unit 100 derives various kinds of information including a plurality of health indexes, a stool state, and the like. Hereinafter, in a case where comprehensively explaining the health indexes and the stool state without differentiating therebetween, it may be referred to as an “index”. For example, indexes include a concept including pieces of information respectively corresponding to the plurality of health indexes of a heart rate, a stress state, a blood circulation state, a fitness level, a body water level, a metabolic level, a blood vessel age, a biological clock, and the like, and further including information corresponding a stool state. In other words, the indexes include various kinds of information to be used in estimating a state related to health of a user, such as pieces of information respectively corresponding to health indexes calculated on the basis of biological information, and information corresponding to a stool state that is determined on the basis of excreted stool information.

For example, the information corresponding to an index may be information that indicates a relative evaluation of the index, for example, “high”, “low”, “equivalent”, and the like. The information corresponding to an index may be a specific numeric value of the index, for example, “50 (bpm)”, “60 (%)” and the like. In a case where clearly indicating that information correspond an index is a numeric value, it may be may be referred to as “score”, “numeric value”, “value”, and the like. In a case where an index is a stool state, information corresponding to the index may be information that indicates a state of a stool, for example, “cracked”, “dark brown”, “middle”, and the like. In a case where an index (may be also referred to “stool index”) is a stool state, for clearly indicating that information corresponding to the index is a numeric value, it may be may be referred to as “excreted stool score”.

The control unit 100 calculates a health score by using information on various indexes such as a health index and a stool state. Note that details of the control unit 100 will be mentioned later.

For example, the communication unit 190 is realized by a communication device, a communication circuit, and the like. For example, the communication unit 190 may be arranged in the main body part 12. The communication unit 190 is connected to an arbitrary network in a wired or wireless manner so as to transmit and receive information to and from an external information processing device. For example, the communication unit 190 transmits and receives information to and from a remote controller, the seating sensor 50, the excreted stool sensor 60, the biological sensor 40, the control unit 100, the external terminal 200, and the like. Note that the communication unit 190 may be included in the control unit 100. In other words, the communication unit 190 may be integrally provided with the control unit 100.

The external terminal 200 displays various kinds of information. The external terminal 200 displays information that is received from the control unit 100. The external terminal 200 includes a display 210 that displays information. For example, the display 210 is a display screen of a tablet terminal and the like, which is realized by, for example, a liquid crystal display, an organic Electro-Luminescence (EL) display, and the like. For example, the external terminal 200 causes the display 210 to display various indexes such as a health index and an index (may be also referred to “stool index”) related to an excreted stool, and various kinds of information such as a health score.

For example, the external terminal 200 is a device (namely, computer) that is used by a user (namely, user). For example, the external terminal 200 is realized by a smartphone, a mobile telephone, a Personal Digital Assistant (PDA), a tablet terminal, a laptop Personal Computer (PC), or the like. For example, the external terminal 200 is a smartphone (namely, portable terminal) that is used by a user.

For example, the external terminal 200 is connected to the control unit 100 via the communication unit 190 to be able to communicate with each other in a wired or wireless manner. For example, the external terminal 200 may be connected to the control unit 100 to be able to communicate with each other by a predetermined wireless communication function such as Bluetooth and Wi-Fi. The external terminal 200 transmits and receives information to and from the control unit 100.

The external terminal 200 receives a content indicating various kinds of information from the control unit 100, and further displays the received content. For example, the external terminal 200 receives a content including a health score from the control unit 100, and further displays the received content. For example, the external terminal 200 executes a process for displaying various kinds of information associated with health, such as a content including a health score, by using application (may be also referred to “health managing application”) for displaying various kinds of information associated with health, such as the content including the health score.

Note that the above-mentioned is merely one example, an arbitrary device configuration may be employed for the health managing system 1 as long as desired processing can be realized. For example, a remote controller may function as a display that displays information. Both of a remote controller and the external terminal 200 may be included in the health managing system 1 as a device that functions as a display. As described above, the above-mentioned system configuration is merely one example, and an arbitrary system configuration may be employed for the health managing system 1 as long as desired processing is possible.

The health managing system 1 may include a sensor other than the biological sensor 40, the seating sensor 50, and the excreted stool sensor 60. For example, the health managing system 1 may include a human body detecting sensor. The human body detecting sensor has function for detecting a human body. For example, the human body detecting sensor is realized by a pyroelectric sensor using an infrared ray signal, and the like. For example, the human body detecting sensor may be realized by a micro (u) wave sensor and the like. Note that the above-mentioned is merely one example, the human body detecting sensor is not limited to the above-mentioned, and may detect a human body by various methods. For example, the human body detecting sensor detects a person (namely, user, etc.) entering a space (namely, toilet room) in which the closet bowl 4 is arranged. The human body detecting sensor transmits a detection signal to the control unit 100.

<1-2. Configuration of Control Unit (namely, Information Processing Device)>

Herein, details of a configuration of the control unit 100 will be explained. The control unit 100 may be an information processing device (namely, controller) configured to control various configurations and processes, for example. For example, the control unit 100 may include a Central Processing Unit (CPU), a Graphics Processing Unit (GPU), or the like to be realized by a program (for example, information processing program according to the present disclosure, etc.), which is stored in the control unit 100, being executed while using RAM and the like as a work region. Furthermore, for example, the control unit 100 may include an integrated circuit such as an Application Specific Integrated Circuit (ASIC) and a Field Programmable Gate Array (FPGA).

As illustrated in FIG. 3, the control unit 100 includes an acquisition unit 110, a storage 120, a health index calculating unit 130, an excreted stool information calculating unit 140, a health state calculating unit 150, and a display processing unit 160 so as to realize or execute functions and working of information processing to be described below. Note that an inner configuration of the control unit 100 is not limited to one illustrated in FIG. 3, and another configuration may be employed as long as the configuration executes information processing to be mentioned later. Note that connection relation between units included in the control unit 100 is not limited to the connection relation illustrated in FIG. 3, and another connection relation may be employed.

The acquisition unit 110 acquires information. The acquisition unit 110 acquires various kinds of information from the storage 120. The acquisition unit 110 acquires various kinds of information from the toilet 10. The acquisition unit 110 acquires, from the toilet 10, various kinds of information collected in a space (namely, toilet room) corresponding to the toilet 10. The acquisition unit 110 acquires, from the toilet 10, user identifying information that identifies a user. The acquisition unit 110 acquires biological information from the toilet 10. The acquisition unit 110 acquires excreted stool information from the toilet 10.

The acquisition unit 110 acquires, from a sensor, sensor information detected by the sensor. The acquisition unit 110 acquires sensor information on a user, which is sensed by a sensor. The acquisition unit 110 acquires biological information on a user. The acquisition unit 110 acquires blood flow information on a user as biological information on the user. The acquisition unit 110 acquires excreted stool information corresponding to an excreted stool of a user. The acquisition unit 110 receives, from the biological sensor 40, biological information that is acquired by the biological sensor 40. The acquisition unit 110 receives, from the seating sensor 50, information that indicates detection by the seating sensor 50. The acquisition unit 110 receives, from the excreted stool sensor 60, excreted stool information that is acquired by the excreted stool sensor 60. The acquisition unit 110 receives, from a remote controller, various kinds of information that indicates an operation of a user and the like. The acquisition unit 110 stores the received various kinds of information in the storage 120.

For example, the storage 120 is realized by a semiconductor memory element such as a Random Access Memory (RAM) and a flash memory, or a storage such as a hard disk and an optical disk. For example, the storage 120 is a non-transitory computer-readable recording medium having stored therein data to be used by an information processing program and the like. The storage 120 stores therein various kinds of information such as information detected by a detection unit, and the storage 120 stores therein various kinds of information to be used in determination processes.

The storage 120 stores therein information related to a health score. The storage 120 stores therein various kinds of information to be used in a calculating process related to a health score. The storage 120 stores therein information on an arithmetic function (namely, health score calculating arithmetic function) to be used in a calculating process of a health score and the like.

The storage 120 stores therein a history (namely, health score history information) related to a health score for each user. The storage 120 stores, as health score history information, information related to a health score associated with an acquired date and hour. The storage 120 stores therein a health score of each of users in association with a corresponding user. For example, the storage 120 stores therein a health score of the user U1 in association with user identifying information for identifying the user U1. Note that the above-mentioned is merely one example, the storage 120 stores therein various kinds of information related to a health score.

The storage 120 stores therein information related to various indexes such as a health index and an index (stool index) related to an excreted stool. The storage 120 stores therein various kinds of information to be used in a calculating process related to a health index.

The storage 120 stores therein information on an arithmetic function (namely, health index calculating arithmetic function) to be used in a calculating process of a health index and the like. The storage 120 stores therein various kinds of information to be used in a calculating process related to an excreted stool score (namely, stool index). The storage 120 stores therein information on an arithmetic function (excreted stool score calculating arithmetic function) to be used in a calculating process of an excreted stool score and the like.

The storage 120 stores therein a history (namely, health index history information) related to a health index for each user. The storage 120 stores therein, as health index history information, information related to a health index associated with an acquired date and hour. The storage 120 stores therein information related to health indexes of each user in association with the corresponding user. For example, the storage 120 stores therein information related to health indexes of the user U1 in association with user identifying information for identifying the user U1. Note that the above-mentioned is merely one example, the storage 120 stores therein various kinds of information related to health indexes.

The storage 120 stores therein various kinds of information to be used in a determination process related to a state of a stool such as a property of a stool. For example, the storage 120 stores therein a threshold to be used in a determination process related to a stool. For example, the storage 120 stores therein various models (namely, determination models) to be used in determination related to a stool. For example, the storage 120 stores therein various determination models to be used in determination of a type, a color, and an amount of a stool and the like.

The storage 120 stores therein a history (namely, stool history information) related to a stool state for each user. The storage 120 stores therein, as stool history information, information related to a stool state associated with an acquired date and hour. The storage 120 stores therein, in association with each user, a type (namely, shape) of a stool, a color of a stool, an amount of a stool in each excreted stool of the corresponding user, and the like. For example, the storage 120 stores therein, in association with user identifying information for identifying the user U1, a type of a stool, a color of a stool, an amount of a stool in each excreted stool of the user U1, and the like. Note that the above-mentioned is merely one example, the storage 120 stores therein various kinds of information related to a stool.

Note that the storage 120 may store therein, not limited to the above-mentioned, various kinds of information in accordance with aims. The storage 120 may store therein biological information and/or excreted stool information. The storage 120 may store therein biological information, excreted stool information, and the like in association with an acquired date and hour thereof. The storage 120 may store therein a stool image as excreted stool information. The storage 120 stores therein information on a stool corresponding to a stool image in association with the stool image. The storage 120 stores therein a determination result (for example, type, color, amount, etc.) determined with respect to a stool corresponding to a stool image in association with the stool image. The storage 120 stores therein information on a property of a stool corresponding to a stool image, an amount of a stool corresponding to a stool image, and the like. The storage 120 may store therein, in association with a stool image, a date and hour when the stool image is acquired, information for identifying a user that has excreted a stool corresponding to the stool image, and the like.

The health index calculating unit 130 functions as a generation unit that generates various kinds of information related to a health index. The health index calculating unit 130 calculates a health index of a user on the basis of a quantification result of the biological sensor 40. For example, the health index calculating unit 130 performs Fourier transform and the like on an output signal of the biological sensor 40 so as to calculate a plurality of health indexes such as a pulse wave, a blood flow volume, and heart rate. The health index calculating unit 130 calculates health index information on the basis of biological information. The health index calculating unit 130 calculates a health index on the basis of biological information. The health index calculating unit 130 calculates a health index score on the basis of biological information. The health index calculating unit 130 calculates health index information on the basis of blood flow information on a user. The health index calculating unit 130 calculates a health index score obtained by quantifying health index information. The health index calculating unit 130 calculates a plurality of pieces of health index information. The health index calculating unit 130 calculates a plurality of pieces of index information on the basis of sensor information.

The health index calculating unit 130 calculates a heart rate on the basis of a quantification result of the biological sensor 40. The health index calculating unit 130 calculates a stress state (namely, calmness level) on the basis of a quantification result of the biological sensor 40. The health index calculating unit 130 calculates a blood circulation state on the basis of a quantification result of the biological sensor 40. The health index calculating unit 130 calculates a fitness level on the basis of a quantification result of the biological sensor 40. The health index calculating unit 130 calculates a body water level on the basis of a quantification result of the biological sensor 40. The health index calculating unit 130 calculates a metabolic level on the basis of a quantification result of the biological sensor 40. The health index calculating unit 130 calculates a blood vessel age on the basis of a quantification result of the biological sensor 40. The health index calculating unit 130 calculates a biological clock on the basis of a quantification result of the biological sensor 40. Note that the health index calculating unit 130 may generate information on a health index by any method as long as being capable of generating the above-mentioned information on various health indexes. For example, the health index calculating unit 130 generates information on various health indexes from a quantification result of the biological sensor 40 by appropriately using various technologies related to health care.

The excreted stool information calculating unit 140 functions as a generation unit that generates various kinds of information related to a stool. The excreted stool information calculating unit 140 functions as an excreted stool state determining unit that executes a determination process of a stool state. In other words, the excreted stool information calculating unit may be replaced with the excreted stool state determining unit. The excreted stool information calculating unit 140 executes a determination process by using information that is detected by the excreted stool sensor 60. The excreted stool information calculating unit 140 executes a determination process by using information stored in the storage 120. The excreted stool information calculating unit 140 determines an excreted stool property of an excreted stool corresponding to excreted stool information on the basis of excreted stool information acquired by the acquisition unit 110. The excreted stool information calculating unit 140 determines an excreted stool property including a type, an amount, and a color of an excreted stool.

The excreted stool information calculating unit 140 determines an amount of a stool on the basis of an image captured by the excreted stool sensor 60. For example, the excreted stool information calculating unit 140 determines an amount of a stool on the basis of an occupied area and/or a ratio of a stool with respect to an image. For example, the excreted stool information calculating unit 140 may determine an amount of a stool by using a score that is output from a stool determining model. In a case where a score output from the stool determining model to which an image has been input is equal to or more than a first threshold, and further is less than a second threshold; the excreted stool information calculating unit 140 may determine an amount of a stool to be “little (small)”. Assume that the second threshold is a value that is larger than the first threshold. In a case where a score output from the stool determining model to which an image has been input is equal to or more than the second threshold, and further is less than a third threshold; the excreted stool information calculating unit 140 may determine an amount of a stool to be “middle”. Assume that the third threshold is a value that is larger than the second threshold. In a case where a score output from the stool determining model to which an image has been input is equal to or more than the third threshold, and further is less than a fourth threshold; the excreted stool information calculating unit 140 may determine an amount of a stool to be “much (large)”. Assume that the fourth threshold is a value that is larger than the third threshold. Note that the above-mentioned three-stage determination is merely one example, and the excreted stool information calculating unit 140 may determine an amount of a stool by appropriately using various kinds of information.

On the basis of an image captured by the excreted stool sensor 60, the excreted stool information calculating unit 140 determines a type of a stool corresponding to a stool image. The excreted stool information calculating unit 140 determines, by using a stool image, that a type of a stool corresponding to the stool image is which of multi-stage types based on shapes (simply referred to as “shape”). The excreted stool information calculating unit 140 may classify a type of a stool into seven types on the basis of the Bristol stool scale. For example, the excreted stool information calculating unit 140 determines, by using a stool image, a type of a stool corresponding to the stool image is which of type 1 to type 7 based on the Bristol stool scale. For example, type 1 is separate-hard, type 2 is stiff (namely, hard), and type 3 is cracked (namely, slightly-hard). Type 4 is normal (namely, smooth and soft sausage), type 5 is slightly-soft, type 6 is mushy, and type is liquid (namely, watery). The excreted stool information calculating unit 140 determines a type of a stool (namely, shape) of a user by appropriately using various technologies for detecting a shape of a stool by an optical method.

The excreted stool information calculating unit 140 may determine a type of a stool by using a technology related to artificial intelligence (AI). For example, the excreted stool information calculating unit 140 may determine a type of a stool by using a learning model (namely, type determining model) that is generated by machine learning. In this case, learning is preliminarily executed on the type determining model by using teacher data that indicates classification determination. The teacher data includes a plurality of combination each of which is constituted of a stool image and a label (namely, correct answer information) that indicates a type (namely, one of type 1 to type 7) of a lump (namely, stool) included in the stool image. For example, the type determining model is a model to which a stool image is input, and that outputs information indicating a type of a lump (namely, stool) included in the input stool image. For example, learning is executed on the type determining model such that in a case where a stool image is input to the type determining model, the type determining model outputs information on a label (namely, type of stool) corresponding to the input stool image. The learning of the type determining model is executed by appropriately using various methods related to so-called supervised learning. In this case, the type determining model may be stored in the storage 120, and further the excreted stool information calculating unit 140 may determine a type of a stool by using the type determining model that is stored in the storage 120. For example, the control unit 100 may execute a learning process so as to generate the type determining model. Note that the above-mentioned is merely one example, and the excreted stool information calculating unit 140 may determine a type of a stool by appropriately using various kinds of information.

For example, by using a stool image, the excreted stool information calculating unit 140 determines a color of a stool corresponding to the above-mentioned stool image. By using a stool image, the excreted stool information calculating unit 140 determines that a color of a stool corresponding to the above-mentioned stool image is which of multistage levels based on a color. For example, by using a stool image, the excreted stool information calculating unit 140 determines that a color of a stool corresponding to the above-mentioned stool image is which of yellow, light ochre, ochre, brown, dark brown (namely, dark brown), and deep dark brown (namely, deep dark brown).

The excreted stool information calculating unit 140 determines a color of a stool on the basis of a detection result obtained by the excreted stool sensor 60. The excreted stool information calculating unit 140 determines a color of a stool of a user by appropriately using various technologies for detecting a color of a stool by an optical method. The excreted stool information calculating unit 140 determines that a color of a stool is which of yellow, light ochre, ochre, brown, dark brown, and deep dark brown by appropriately using various technologies related to classification of a color of a stool. For example, the excreted stool information calculating unit 140 determines (namely, determines) a color of a stool on the basis of various kinds of information (namely, feature amount) such as luminance and brightness of a color image (e.g., RGB).

The excreted stool information calculating unit 140 may determine a color of a stool by using a technology related to artificial intelligence (AI). For example, the excreted stool information calculating unit 140 may determine a color of a stool by using a learning model (namely, color determining model) that is generated by machine learning. In this case, learning is preliminarily executed on the color determining model by using teacher data that indicates classification determination. The teacher data includes a plurality of combination each of which is constituted of a stool image and a label (namely, correct answer information) that indicates a color (one of yellow, light ochre, ochre, brown, dark brown, and deep dark brown) of a lump (namely, stool) included in the stool image. For example, the color determining model is a model to which a stool image is input, and that outputs information indicating a color of a lump (namely, stool) included in the input stool image. For example, learning is executed on the color determining model such that in a case where a stool image is input to the color determining model, the color determining model outputs information on a label (namely, color of stool) corresponding to the input stool image. The learning of the color determining model is executed by appropriately using various methods related to so-called supervised learning. In this case, the color determining model may be stored in the storage 120, and further the excreted stool information calculating unit 140 may determine a color of a stool by using the color determining model that is stored in the storage 120. For example, the control unit 100 may execute a learning process so as to generate the color determining model. Note that the above-mentioned is merely one example, and the excreted stool information calculating unit 140 may determine a color of a stool by appropriately using various kinds of information. Note that the above-mentioned six stages of yellow, light ochre, ochre, brown, dark brown, and deep dark brown are merely examples of a color, and thus the excreted stool information calculating unit 140 may determine another color, or may execute determination by using five or less stages.

As described above, the excreted stool information calculating unit 140 determines a stool state on the basis of excreted stool information. The excreted stool information calculating unit 140 calculates information related to a stool index on the basis of sensor information. The excreted stool information calculating unit 140 determines a stool index on the basis of excreted stool information. The excreted stool information calculating unit 140 calculates an excreted stool score on the basis of a stool state. The excreted stool information calculating unit 140 calculates an excreted stool score obtained by quantifying excreted stool information. The excreted stool information calculating unit 140 calculates an excreted stool score on the basis of relative evaluation obtained by comparison between a property value of a stool excreted in a single excretion of a stool and a property value as a reference.

The health state calculating unit 150 calculates a health score. On the basis of health index information based on biological information, and excreted stool information; the health state calculating unit 150 calculates a health score that is numeric value information indicating a health state of a user. Note that the numeric value information includes various kinds of information such as what percentage, not limited to a point indicating how many points out of 100 points indicating full marks. The health state calculating unit 150 calculates a health score by using health index information that is calculated by the health index calculating unit 130.

The health state calculating unit 150 calculates a health score by using a health index score calculated by the health index calculating unit 130, and an excreted stool score calculated by the excreted stool information calculating unit 140. The health state calculating unit 150 calculates a health score on the basis of an excreted stool score and a health index score. The health state calculating unit 150 calculates a health score on the basis of a plurality of health index scores and an excreted stool score. The health state calculating unit 150 calculates a health score on the basis of a total value obtained by totaling a plurality of health index scores and an excreted stool score.

The health state calculating unit 150 calculates, as a health score, an average value of a plurality of health index scores and an excreted stool score. The health state calculating unit 150 calculates a health score by using a plurality of weight values respectively corresponding to health index scores and an excreted stool score. The health state calculating unit 150 calculates a health score on the basis of a total value obtained by totaling weighed health index scores, which are obtained by respectively multiplying health index scores by weight values corresponding to the health index scores, and a weighed excreted stool score that is obtained by multiplying an excreted stool score by a weight value corresponding to the excreted stool score.

The display processing unit 160 functions as an output controlling unit that controls an output of various kinds of information. The display processing unit 160 controls displaying of the display 210 of the external terminal 200. The display processing unit 160 transmits an instruction to the external terminal 200 so as to control displaying the display 210 of the external terminal 200. The display processing unit 160 transmits information to the external terminal 200 so as to control displaying of the display 210 of the external terminal 200.

The display processing unit 160 transmits information to the external terminal 200 via the communication unit 190. For example, the display processing unit 160 transmits, to the external terminal 200, information to be displayed on the external terminal 200. The display processing unit 160 functions as a determination unit that executes various determinations. The display processing unit 160 executes determination related to time on the basis of time measured by the timer 70. The display processing unit 160 determines whether or not the seating sensor 50 detects sitting of a user. The display processing unit 160 determines whether or not the seating sensor 50 detects sitting of a user on the toilet seat 20.

The display processing unit 160 executes a process for causing the external terminal 200 to display on the display 210 thereof, as time series data, an excreted stool property that is determined by the excreted stool information calculating unit 140. The display processing unit 160 is capable of switching displaying at predetermined time intervals, and further executes a process for causing the display 210 of the external terminal 200 to display, from among excreted stool property patterns during a predetermined time interval, a predetermined number of ones whose frequency is large.

The display processing unit 160 executes a process for switching displaying on the display 210 of the external terminal 200 from among a day unit, a week unit, a month unit, and a year unit. In a case where executing displaying by at least a month unit or a year unit, the display processing unit 160 executes a process for causing the display 210 of the external terminal 200 to display, from among patterns corresponding to excreted stool properties during a predetermined time interval, a predetermined number of patterns whose frequency is large. In a case where executing displaying by at least a day unit, the display processing unit 160 displays all excreted stool properties of a user, which are determined by the excreted stool information calculating unit 140.

The display processing unit 160 executes a process for causing the display 210 of the external terminal 200 to execute displaying on the basis of a pattern corresponding to a combination of a type, an amount, and a color. The display processing unit 160 executes a process for causing the display to display patterns on the basis of a preference order of a type, an amount, and a color in this order. In a case where the number of displayed objects on the display 210 of the external terminal 200 is limited, the display processing unit 160 executes a process for causing the display to display patterns corresponding to the number of displayed objects on the basis of a preference order of a type, an amount, and a color in this order.

The display processing unit 160 executes a process for displaying, on the display 210 of the external terminal 200, information related to a health score that is calculated by the health state calculating unit 150. The display processing unit 160 executes a process for displaying, on the display 210 of the external terminal 200, information related to a health score, information related to health index information, and information related to excreted stool information. The display processing unit 160 executes a process for displaying, on the display 210 of the external terminal 200, information indicating an excreted stool state of a user based on excreted stool information. The display processing unit 160 executes a process for displaying, on the display 210 of the external terminal 200, character information indicating an excreted stool state of a user based on excreted stool information.

The display processing unit 160 executes a process for causing the display 210 of the external terminal 200 to display a health index score. The display processing unit 160 executes a process for causing the display 210 of the external terminal 200 to display a plurality of pieces of health index information. The display processing unit 160 executes a process for causing the display 210 of the external terminal 200 to display a date and hour when at least each of excreted stool information and health index information is finally obtained.

In a case where all of health index information and excreted stool information to be used in calculation of a health score are not obtained, the display processing unit 160 executes a process for hiding a health score from the display 210 of the external terminal 200. In a case where any of health index information and excreted stool information to be used in calculation of a health score is updated, the display processing unit 160 updates the health score.

The display processing unit 160 executes a process for causing the display 210 of the external terminal 200 to display index information that is calculated by the health index calculating unit 130 or the excreted stool information calculating unit 140. In a case where acquisition of sensor information on a user ends after completion (namely, end) of measurement (may be also referred to as “primary measurement”) related to a first index, the display processing unit 160 executes a process for causing the display 210 of the external terminal 200 to display information (may be also referred to as “first index information”) related to the first index.

The display processing unit 160 executes a process for causing the display 210 of the external terminal 200 to display the first index information and information (may be also referred to as “second index information”) related to a second index after an end of measurement (may be also referred to as “secondary measurement”) related to the second index. In a case where first index information is updated during a time interval from an end of the primary measurement until an end of the secondary measurement, the display processing unit 160 executes a process for causing the display 210 of the external terminal 200 to display the updated first index information.

Note that the first index and the second index are relative concepts so as to be prescribed in accordance with a length of a time interval (namely, necessary time interval) needed for acquisition (namely, measurement) of information, and an index whose necessary time interval is longer than that of one index that is the first index becomes the second index with respect to the one index. For example, in a case where a necessary time interval of a stool state (namely, stool index) is longer than that of one health index, and further the one health index is the first index; the stool index becomes a second index with respect to the one health index. For example, in a case where a necessary time interval of the one health index is longer than that of another health indexes, and further the other health index is a first index; the one health index becomes a second index with respect to the other health index. As described above, in a case where different sensors of the biological sensor 40 and the excreted stool sensor 60 are used for calculating a plurality of health indexes, quantification time intervals of the sensors are different from each other. For example, in a case where a long time interval (for example, several minutes and the like) is necessary until the time of excretion of a stool by a user, regarding quantification of a health index by the biological sensor 40 and measurement of an excreted stool state (namely, stool state) by the excreted stool sensor 60, a necessary time interval of the health index is longer than a necessary time interval of the stool state (namely, stool index).

For example, in a case where a necessary time interval of a stool state (namely, stool index) is longer than that of a stress state, and further a stress state is a first index, the stool index is a second index with respect to the stress state. For example, in a case where a necessary time interval until the time of acquisition (namely, determination) of information on a stool state (namely, stool index) is 90 seconds and a necessary time interval until the time of acquisition (namely, calculation) of information on a stress state is 60 seconds, and further the stress state is a first index; the stool index is a second index with respect to the stress state. In a case where a necessary time interval of a stress state is longer than that of a heart rate, and the heart rate is a first index; the stress state is a second index with respect to the heart rate. For example, a necessary time interval until the time of acquisition (namely, calculation) of information on a stress state is 50 seconds and a necessary time interval until the time of acquisition (namely, calculation) of information on a heart rate is 15 seconds, and further the heart rate is a first index; the stress state is a second index with respect to the heart rate. As described above, the first index and the second index are relative concepts, a first index becomes a second index with respect to an index whose necessary time interval is shorter than that of the first index. Even an index, which is a second index with respect to another index, becomes a first index with respect to an index whose necessary time interval is longer than the index. In other words, the above-mentioned first and the second indexes are merely names for differentiating and expressing indexes in accordance with a length of a necessary time interval.

The display processing unit 160 functions as a generation unit that executes a generation process of various kinds of information. The display processing unit 160 generates a content for causing the external terminal 200 to execute displaying. The display processing unit 160 generates a content CT1 that indicates a health score. For example, the display processing unit 160 generates a content (namely, image information) to be provided to the external terminal 200 by appropriately using various technologies related to image generation, image processing, and the like. For example, the display processing unit 160 generates a screen (namely, image information) to be provided to the external terminal 200 by appropriately using various technologies such as Java (Registered Trademark). Note that the display processing unit 160 may generate a content (namely, image information) to be provided to the external terminal 200 on the basis of a format of Cascading Style Sheets (CSS), JavaScript (Registered Trademark), or Hyper Text Markup Language (HTML). For example, the display processing unit 160 may generate a content on the basis of various kinds of format such as Joint Photographic Experts Group (JPEG), Graphics Interchange Format (GIF), and Portable Network Graphics (PNG).

The display processing unit 160 transmits information. The display processing unit 160 transmits information to an external information processing device via the communication unit 190. For example, the display processing unit 160 transmits various kinds of information to the external terminal 200. The display processing unit 160 transmits generated information to the external terminal 200 and the like. The display processing unit 160 transmits the content CTI to the external terminal 200 and the like.

The display processing unit 160 controls displaying by the display 210 of the external terminal 200 such that the content CT1 is displayed on the display 210 of the external terminal 200. Note that the above-mentioned process for displaying on the display 210, which is executed by the display processing unit 160 and the like, includes that the display processing unit 160 and the like transmit information to the external terminal 200 including the display 210 so as to cause the external terminal 200 to display the information.

The display processing unit 160 includes a health state display processing unit 161, a health index display processing unit 162, an excreted stool state display processing unit 163, a measurement situation display processing unit 164, a message display processing unit 165, and a highlight display processing unit 166.

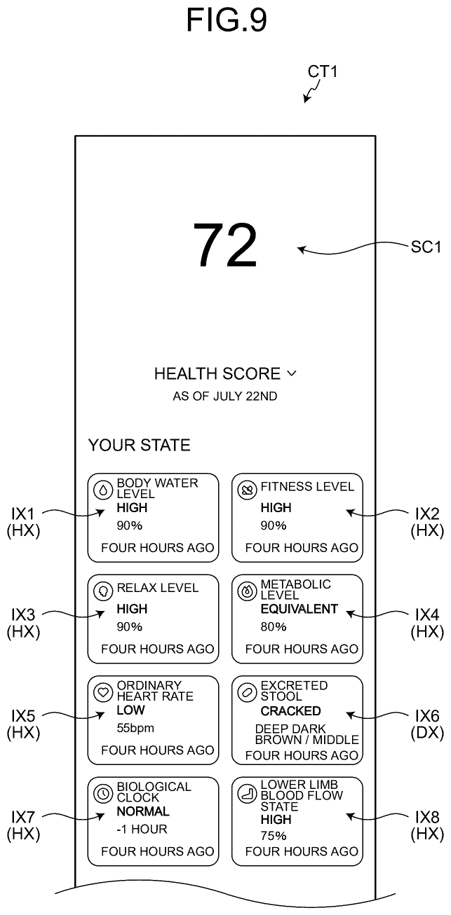

The health state display processing unit 161 executes a process related to displaying of information related to a health score from among processes to be executed by the display processing unit 160. Information to be displayed related to a health score includes information for visually displaying numeric values such as points and percentage by using a gauge bar and the like, in addition to numeric values including, for example, a point indicating how many points out of 100 points indicating full marks, percentage indicating what percentage, and the like. As described above, in a case where a score is displayed, the numeric value may be a numeric value indicating an absolute value (how many points out of 100 points indicating full marks), or may be a numeric value indicating relative percentage. Moreover, instead of numeric value information, a gauge obtained by visualizing the numeric value information and the like may be displayed. The health state display processing unit 161 executes a process for causing the display 210 of the external terminal 200 to display information related to a health score. In a case where a health score SC1 is updated among the content CT1 illustrated in FIG. 9, the health state display processing unit 161 transmits the updated health score SC1 to the external terminal 200 so as to execute a process for causing the external terminal 200 to display the updated health score SC1.

The health index display processing unit 162 executes a process related to displaying of information related to a health index from among processes to be executed by the display processing unit 160. The health index display processing unit 162 executes a process for causing the display 210 of the external terminal 200 to display information related to health index information. In a case where one of health indexes HX is updated from among the content CT1 illustrated in FIG. 9, the health index display processing unit 162 transmits the updated health index HX to the external terminal 200 so as to execute a process for causing the external terminal 200 to display the updated health index HX.

The health index display processing unit 162 executes a process for causing the display 210 of the external terminal 200 to display health index information calculated by the health index calculating unit 130. In a case where acquisition of sensor information on a user ends after end of the primary measurement, the health index display processing unit 162 executes a process for causing the display 210 of the external terminal 200 to display first index information. The health index display processing unit 162 executes, after end of the secondary measurement, a process for causing the display 210 of the external terminal 200 to display first index information and second index information.

The excreted stool state display processing unit 163 executes a process related to displaying of information related to an excreted stool state from among processes to be executed by the display processing unit 160. The excreted stool state display processing unit 163 executes a process for causing the display 210 of the external terminal 200 to display information related to excreted stool information. In a case where a stool index DX is updated from among the content CT1 illustrated in FIG. 9, the excreted stool state display processing unit 163 transmits the updated stool index DX to the external terminal 200 so as to execute a process for causing the external terminal 200 to display the updated stool index DX.

The excreted stool state display processing unit 163 executes a process for causing the display 210 of the external terminal 200 to display stool index information that is calculated by the excreted stool information calculating unit 140. The excreted stool state display processing unit 163 execute a process for causing the display 210 of the external terminal 200 to display second index information after an end of the secondary measurement.

The measurement situation display processing unit 164 executes a process related to displaying of information related to a measurement situation from among processes to be executed by the display processing unit 160. The measurement situation display processing unit 164 executes a process for causing the display 210 of the external terminal 200 to display information related to a measurement situation.

The measurement situation display processing unit 164 causes the display 210 of the external terminal 200 to report a measurement situation. The measurement situation display processing unit 164 causes the display 210 of the external terminal 200 to report a plurality of pieces of index information related to a measurement situation, which includes first index information that can be calculated during the first time interval, and second index information that can be calculated during the second time interval that is longer than the first time interval. The measurement situation display processing unit 164 executes a completion notification of the primary measurement corresponding to the first time interval, and further executes an execution notification of the secondary measurement corresponding to the second time interval. In a case where the secondary measurement ends, the measurement situation display processing unit 164 executes a completion notification of the secondary measurement.

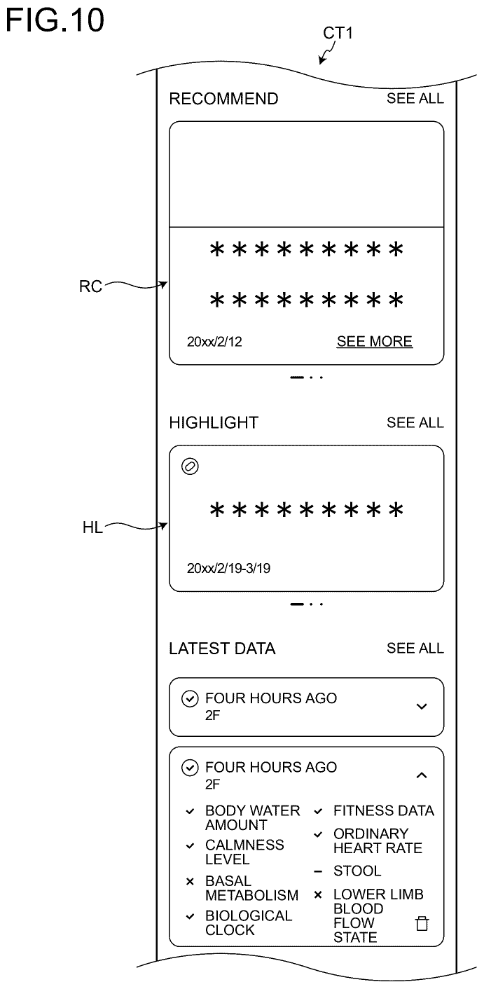

On the basis of at least one of a health score, health index information, and excreted stool information, the message display processing unit 165 determines a state of a user so as to execute a process for causing the display 210 of the external terminal 200 to display a recommendation message according to the determined state of the user. For example, the message display processing unit 165 executes a process for causing the display 210 of the external terminal 200 to display recommendation information RC illustrated in FIG. 10.

On the basis of at least one of a health score, health index information, and excreted stool information; the highlight display processing unit 166 executes a process for executing highlight display on the display 210 of the external terminal 200 for a predetermined time interval. For example, the highlight display processing unit 166 executes a process for causing the display 210 of the external terminal 200 to display highlight information HL illustrated in FIG. 10.

Note that the above-mentioned configuration of the control unit 100 is merely one example, and not limited to the above-mentioned, the control unit 100 may have various configurations. For example, in a case where having a function for displaying information, the control unit 100 may include a display. In a case where the external terminal 200 calculates a health score, the control unit 100 and the external terminal 200 may be integrated with each other. For example, in a case where the external terminal 200, such as a smartphone used by a user, calculates a health score by using an information processing program; the external terminal 200 may have function of the control unit 100.

For example, a process may be executed, which is for calculating a health score by using application (for example, health managing application) including an information processing program. For example, in a case where calculating a health score by using application (for example, health managing application) that is installed in the external terminal 200, the external terminal 200 may include a configuration of the control unit 100.

### <2. Calculation Example>

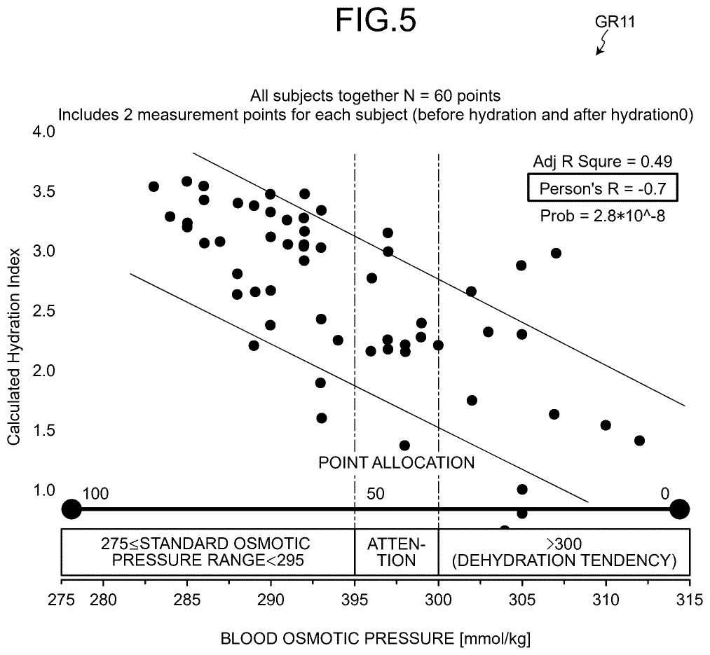

Hereinafter, one example of a calculation method of a numeric value will be explained with reference to FIG. 5 to FIG. 7. FIG. 5 to FIG. 7 are diagrams illustrating examples of calculation methods of numeric values. Hereinafter, calculation of a numeric value (namely, score) will be explained while exemplifying a body water amount as one example.

<2-1. First Calculation Example (namely, Point Allocation)>

First, a first calculation example will be explained with reference to FIG. 5. Specifically, FIG. 5 illustrates the first calculation example for allocating (namely assigning) a point so as to calculate a score. A graph GR11 illustrated in FIG. 5 indicates information for converting (converting) a measurement result into 0 to 100 points. Specifically, the graph GR11 indicates an example in which a point (namely, score) corresponding to a body water amount is allocated to a quantification value (namely, measurement value) corresponding to the body water amount. In the example illustrated in FIG. 5, a case is exemplified in which a point to be allocated is larger as a quantification value corresponding to a body water amount is smaller. In this case, by using information on the graph GR11 and based on a quantification value corresponding to the quantified body water amount, the control unit 100 calculates a health index score (namely, body water amount score) corresponding to the body water amount.

<2-2. Second Calculation Example (for each User)>

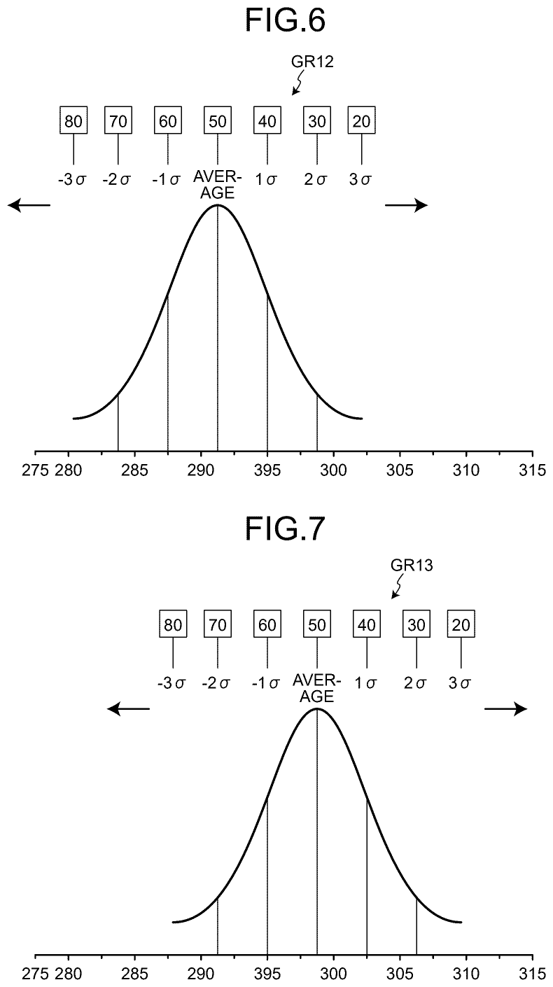

A second calculation example will be explained with reference to FIG. 6. FIG. 6 illustrates an example in which calculation is executed based on own result of health index information. Specifically, FIG. 6 illustrates the second calculation example for executing point allocation (namely, assignment) so as to calculate a score. FIG. 6 illustrates the second calculation example in which a score is calculated by using a history of a user (namely, target user) alone to be a calculation target. For example, FIG. 6 illustrates a case where a point is allocated on the basis of past 50 measurement results of a target user.

A graph GR12 illustrated in FIG. 6 indicates information for converting (namely, converting) a measurement result into 0 to 100 points. For example, the graph GR12 is information for converting (namely, converting) a measurement result into a value between 0 to 100 points in accordance with a discrepancy from a base while regarding an average value (namely, base) as 50 points. Specifically, the graph GR12 indicates an example in which a point (namely, score) corresponding to a body water amount is allocated to a quantification value (namely, measurement value) corresponding to the body water amount. In the example illustrated in FIG. 6, a case is exemplified in which a point to be allocated is larger as a quantification value corresponding to a body water amount is smaller. In this case, by using information on the graph GR12 and on the basis of a quantification value corresponding to the quantified body water amount, the control unit 100 calculates a health index score (namely, body water amount score) corresponding to the body water amount.

### <2-3. Third Calculation Example (Plurality of Users)>

With reference to FIG. 7, a third calculation example will be explained. FIG. 7 illustrates an example in which calculation is executed on the basis of result of health index information on a user population (namely, plurality Specifically, FIG. 7 illustrates the third of users). calculation example for executing point allocation (namely, assignment) so as to calculate a score. FIG. 7 illustrates the third calculation example in which a score is calculated by using histories of a plurality of users, not limited to a user (namely, target user) to be a calculation target. For example, FIG. 7 illustrates a case where a point is allocated on the basis of past 50 measurement results of each of 100 users.

A graph GR13 illustrated in FIG. 7 indicates information for converting (namely, converting) a measurement result into 0 to 100 points. For example, the graph GR13 is information for converting (namely, converting) a measurement result into a value between 0 to 100 points in accordance with a discrepancy from a base while regarding an average value (namely, base) as 50 points. Specifically, the graph GR13 indicates an example in which a point (namely, score) corresponding to a body water amount is allocated to a quantification value (namely, measurement value) corresponding to the body water amount. In the example illustrated in FIG. 7, a case is exemplified in which a point to be allocated is larger as a quantification value corresponding to a body water amount is smaller. In this case, by using information on the graph GR13 and on the basis of a quantification value corresponding to the quantified body water amount, the control unit 100 calculates a health index score (namely, body water amount score) corresponding to the body water amount.

Note that the above-mentioned calculation is merely one example, the control unit 100 may calculate a numeric value by various methods, not limited to the above-mentioned. For example, the control unit 100 may calculate a numeric value by an absolute value evaluation. In FIG. 5 to FIG. 7, calculation of a numeric value (namely, score) is explained while exemplifying a body water amount as one example which is one of health indexes; a health index score may be calculated by a similar process with respect to another health index such as a heart rate.

The control unit 100 may calculate an excreted stool score by using a method that is similar to those illustrated in FIG. 5 to FIG. 7. For example, the control unit 100 calculates an excreted stool score having a value within the range of 0 to 100 points. For example, the control unit 100 may calculate an excreted stool score on the basis of relative evaluation obtained by comparison between a property value of a stool that is excreted in a single excretion of a stool and a property value as a reference. For example, the control unit 100 calculates an excreted stool score on the basis of comparison between a reference, which is average of stool states of past stools of a user, and a stool state of a stool (may be also referred to as “target stool”) to be a calculation target. Note that as described above, the excreted stool score may include a numeric value calculated on the basis of an excreted stool property (namely, stool state) such as a type, a shape, and a color; or may include information indicating an excreted stool property (namely, stool state) itself, which indicates a type, a shape, and a color.

For example, the control unit 100 calculates an excreted stool score whose value is larger as a stool state of a target stool is closer to a reference. For example, the control unit 100 calculates an excreted stool score of a target stool such that a value thereof is smaller as the stool state of the target stool is farther from a reference while regarding a case where a stool state of a target stool coincides with the reference as 100 points. For example, in a case where a stool state of a target stool coincides with a reference, the control unit 100 calculates an excreted stool score of the target stool as 100 points. For example, the control unit 100 calculates an excreted stool score of a target stool to be smaller as a stool state of the target stool is farther from a reference.

For example, the control unit 100 may execute plotting with respect to a stool on a three-dimensional space (may be also referred to as “stool state space”) whose dimensions are a type, a color, and an amount, respectively. An excreted stool score may be calculated in accordance with a position of the stool in the three-dimensional space (namely, stool state space). For example, in accordance with where a stool is located in a stool state space whose dimensions are a type, a color, and an amount, respectively; the control unit 100 may calculate an excreted stool score of the stool. For example, the control unit 100 may set, as an excreted stool score of the stool, a point corresponding to a part where a stool is located in the stool state space whose dimensions are a type, a color, and an amount, respectively, and in which points are allocated to respective positions.

### <3. Flow of Processing>

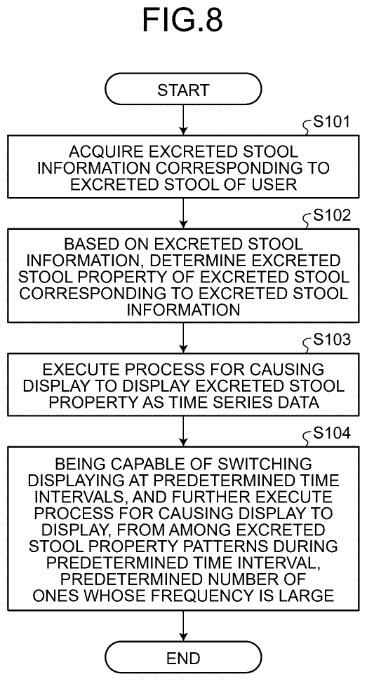

Hereinafter, a processing flow related to the embodiment will be explained with reference to FIG. 8. FIG. 8 is a flowchart illustrating one example of a procedure for processes to be executed by the health managing system. Specifically, FIG. 8 is a flowchart illustrating the outline of a procedure for processes related to excreted stool information, which are to be executed by the health managing system. Hereinafter, the health managing system 1 will be explained as a processing subject; however, processes illustrated in FIG. 8 may be executed by any of the control unit 100, the external terminal 200, various sensors, and the like in accordance with a device configuration included in the health managing system 1.

The outline of a processing procedure related to excreted stool information of the health managing system 1 will be explained with reference to FIG. 8.

The health managing system 1 acquires excreted stool information corresponding to an excreted stool of a user (Step S101). For example, the excreted stool sensor 60 of the health managing system 1 acquires excreted stool information corresponding to an excreted stool of a user that is using the toilet 10.

On the basis of the excreted stool information, the health managing system 1 determines an excreted stool property of the excreted stool corresponding to the excreted stool information (Step S102). For example, by using excreted stool information acquired by the excreted stool sensor 60, the health managing system 1 determines an excreted stool property of a stool corresponding to the excreted stool information.

The health managing system 1 executes a process for causing a display to display an excreted stool property as time series data (Step S103). For example, the control unit 100 of the health managing system 1 executes a process for causing the display 210 of the external terminal 200 to display an excreted stool property as time series data. For example, the control unit 100 transmits information indicating a stool property in a time series to the external terminal 200 including the display 210. The external terminal 200, which has received the information indicating a stool property in a time series from the control unit 100, causes the display 210 to display information that indicates the stool property in a time series.

The health managing system 1 is capable of switching displaying at predetermined time intervals, and further executes a process for causing a display to display, from among excreted stool property patterns during a predetermined time interval, a predetermined number of ones whose frequency is large (Step S104). For example, the control unit 100 of the health managing system 1 is capable of switching displaying at a plurality of time intervals of a day, a week, a month, a year, and the like; and further executes a process for causing the display 210 of the external terminal 200 to display, from among excreted stool property patterns during a time interval to be displayed of a day, a week, a month, a year, and the like, a predetermined number of ones whose frequency is large. For example, the control unit 100 transmits a content that indicates, in a time series, a stool property whose displaying can be switched between a plurality of time intervals such as a day, a week, a month, and a year; to the external terminal 200 including the display 210. The external terminal 200 causes the display 210 to display a content that indicates, in a time series, a stool property whose displaying can be switched between a plurality of time intervals such as a day, a week, a month, and a year.

### <4. Displaying Example>

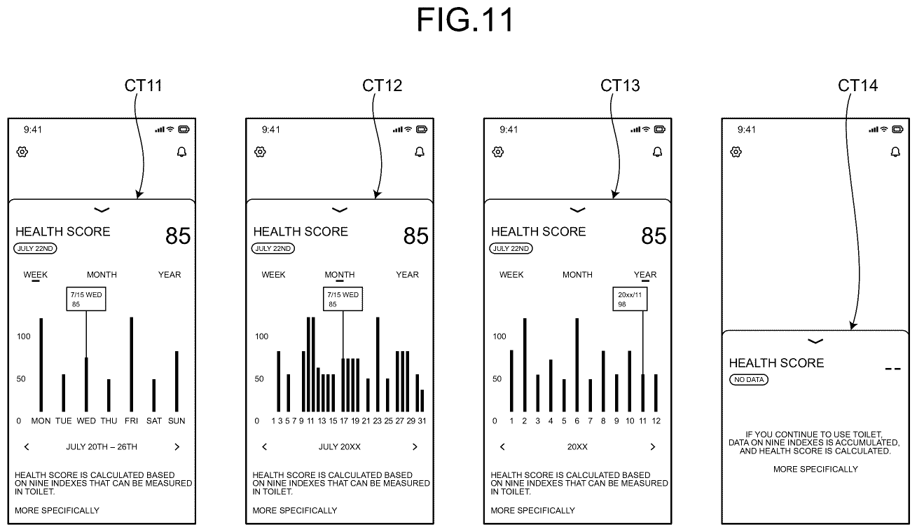

Hereinafter, a displaying example of various kinds of information will be explained with reference to FIG. 9 to FIG. 16. First, allover displaying examples of a health score and the like will be explained with reference to FIG. 9 to FIG. 11. FIG. 9 is a diagram illustrating a displaying example of a health score. FIG. 10 is a diagram illustrating a displaying example of information associated with health. FIG. 11 is a diagram illustrating a displaying example according to a time interval. Hereinafter, a case will be explained as one example in which a target user is the user U1, and information is displayed on the external terminal 200 used by the user U1, such as a smartphone.

<4-1. Displaying Example of Health Score and the like>

A displaying example of a health score and the like will be explained with reference to FIG. 9. For example, the control unit 100 causes the external terminal 200 used by the user U1 to display information that indicates a health score, a health index, and a stool state regarding the user U1. The control unit 100 transmits information that indicates a health score, a health index, and a stool state regarding the user U1 to the external terminal 200 used by the user U1. The external terminal 200 used by the user U1 displays information that indicates a health score, a health index, and a stool state received from the control unit 100.

In FIG. 9, the control unit 100 generates the content CT1 including information related to a health score, information related to health index information, and information related to excreted stool information regarding the user U1. Specifically, the control unit 100 generates the content CT1 including information that indicates indexes IX1 to IX8 of the health score SC1, a health index, and a stool state of the user U1 and the like.

The indexes IX1 to IX5, IX7, and IX8 correspond to the health indexes HX, and the index IX6 corresponds to the stool index DX. Specifically, the index IX1 corresponds to a body water level, the index IX2 corresponds to a fitness level, the index IX3 corresponds to a relax level (namely, stress state), and the index IX4 corresponds to a metabolic level. The index IX5 corresponds to a heart rate (namely, ordinary heart rate), the index IX6 corresponds to a stool state, the index IX7 corresponds to a biological clock, and the index IX8 corresponds to a blood circulation state (namely, lower limb blood flow state).

In explanation without differentiating between the index IX1 to IX5, IX7, and IX8, they may be referred to as “health indexes HX”, and in explanation without differentiating the index IX6, it may be referred to as “stool index DX”. In explanation without differentiating between the indexes IX1 to IX8, they may be referred to as the “indexes IX”. Note that the above-mentioned indexes IX1 to IX8 are merely one example, and an arbitrary index such as an index IX9 corresponding to a blood vessel age may be included.

The control unit 100 generates the content CT1 that includes the health score SC1 of the user U1 and information indicating a date and hour when each of the indexes IX1 to IX8 is obtained. For example, the control unit 100 calculates a health score at a predetermined timing (once per day and the like). The control unit 100 calculates the health score SC1 that is “72” points illustrated in FIG. 9 on Jul. 22, 2022. The control unit 100 calculates the health score SC1 of “72” points by using scores corresponding to the indexes IX. The control unit 100 calculates the health score SC1 of “72” points by using health index scores of the indexes IX1 to IX5, IX7 and IX8, and an excreted stool score of the index IX6. The control unit 100 generates the content CT1 including information indicating that the health score SC1 of “72” points illustrated in FIG. 9 is calculated on July 22.

For example, the control unit 100 calculates information on the indexes at a timing when sensor information is acquired from sensors. For example, the control unit 100 calculates information on the indexes IX1 to IX8 four hours before generation of the content CT1. The control unit 100 generates the content CT1 including information indicating that information on the indexes IX1 to IX8 illustrated in FIG. 9 is calculated four hours ago.

Note that information indicating a date and hour corresponding to each of the indexes IX1 to IX8 may be a date and hour when sensor information corresponding to each of the indexes IX1 to IX8 is acquired.

In FIG. 9, regarding the health indexes HX of the indexes IX1 to IX5, IX7, and IX8; the control unit 100 generates the content CT1 including information on relative evaluation of information (may be also referred to as “first information”) that is used in calculating the health score SC1, with respect to predetermined references (may be also referred to as “evaluation criteria”). For example, regarding the user U1, the control unit 100 generates the content CT1 including information on relative evaluation with respect to an evaluation criterion that is calculated by using information (may be also referred to as “second information”) prior to the first information.

In FIG. 9, regarding the index IX1, the control unit 100 generates the content CT1 including information indicating that a body water level is “90%”, and first information used in calculating the health score SC1 is higher than an evaluation criterion such as a past average. Regarding the index IX5, the control unit 100 generates the content CT1 including information indicating that a heart rate is “55 bpm”, and first information used in calculating the health score SC1 is lower than an evaluation criterion such as a past average.

Regarding the index IX6 that is the stool index DX, the control unit 100 generates the content CT1 including information indicating a type “cracked”, a color “deep dark brown”, and an amount “middle”. Note that the above-mentioned is merely one example, the control unit 100 may generate the content CT1 that includes various kinds of information. For example, the control unit 100 may generate the content CT1 including a health index score as information on each of the health indexes HX. For example, the control unit 100 may generate the content CT1 including an excreted stool score as information on the stool index DX.

The control unit 100 transmits the content CT1 to the external terminal 200, and the external terminal 200 displays the received content CT1. The external terminal 200 displays the content CT1 including information related to the health score SC1 of the user U1, information related to the health index HX of the user U1, and information related to the stool index DX of the user U1. Note that the health managing system 1 may display a health index score as information on each of the health indexes HX.

In FIG. 9, a case is exemplified as one example where information on all indexes ID to be used in calculating the health score SC1 has already been acquired; however, in a case where information on at least one of all indexes ID to be used in calculating the health score SC1 has not been acquired, the control unit 100 may hide the health score SC1. For example, in a case where information on at least one of all indexes ID to be used in calculating the health score SC1 has not been acquired, the control unit 100 generates the content CT1 in which information (for example, “−” and the like) indicating that a corresponding health score is not calculated is arranged in a position of the health score SC1. The control unit 100 transmits, to the external terminal 200, the content CT1 in which “−” is arranged in a position corresponding to the health score SC1 so as to hide the health score SC1; and the external terminal 200 displays the content CT1 in which the health score SC1 is hidden. The external terminal 200 displays the content CT1 in which “−” is arranged in a position corresponding to the health score SC1, and further the health score SC1 is hidden. In a case where information on all indexes ID to be used in calculating the health score SC1 has been acquired, the health managing system 1 displays the content CT1 including the calculated health score SC1 as illustrated in FIG. 9.

### <4-2. Displaying Example of Recommendation and Highlight>

Next, with reference to FIG. 10, a displaying example of recommendation (approval) and highlight will be explained.

For example, regarding the user U1, on the basis of at least one of a health score, health index information, and excreted stool information; the control unit 100 determines a state of the user U1 so as to cause the external terminal 200 to display a recommendation message according to the determined state of the user. The control unit 100 transmits, to the external terminal 200 used by the user U1, a recommendation message that is generated in accordance with a state of the user U1. The external terminal 200 used by the user U1 displays a recommendation message that is received from the control unit 100.

For example, in a case where a biological clock of the user U1 deviates by equal to or more than a predetermined threshold, the control unit 100 determines that a state of the user U1 is a shortage of sleep so as to generate a recommendation message RC1 that urges the user U1 to have appropriate sleeping. In FIG. 10, the control unit 100 generates the content CT1 including the recommendation message RC1 that urges the user U1 to have appropriate sleeping.

For example, regarding the user U1, on the basis of at least one of a health score, health index information, and excreted stool information; the control unit 100 causes the external terminal 200 used by the user U1 to execute highlight displaying for a predetermined time interval (for example, one hour, one day, and the like).

On the basis of at least one of a health score, health index information, and excreted stool information; the control unit 100 transmits the generated highlight information to the external terminal 200 used by the user U1. The external terminal 200 used by the user U1 displays the highlight information that is received from the control unit 100.

For example, in a case where a ratio where excretion of a stool by the user U1 is performed by 9 p.m. is equal to or more than a predetermined threshold during a predetermined time interval (for example, one month and the like), the control unit 100 generates highlight information HL1 indicating that an excretion rhythm of a stool in the user U1 is appropriate. In FIG. 10, the control unit 100 generates the content CT1 including the highlight information HL1 indicating that an excretion of a stool in the user U1 is appropriate.

The control unit 100 transmits the content CT1 to the external terminal 200, and the external terminal 200 displays the received content CT1. The external terminal 200 displays the content CT1 that includes the recommendation message RC1 for the user U1 and the highlight information HL1.

### <4-3. History Displaying Example of Health Score>

Note that in FIG. 9, a case is exemplified as one example in which the last calculated health score SC1 alone is displayed, the health managing system 1 may execute displaying based on a history of a health score. The above-mentioned point will be explained with reference to FIG. 11.

For example, regarding the user U1, the control unit 100 causes the external terminal 200 used by the user U1 to display time series information (may be also referred to as “health score time series”) based on a history of a health score that is calculated during a predetermined time interval. Regarding the user U1, the control unit 100 transmits, to the external terminal 200 used by the user U1, information indicating a health score time series obtained by calculating past calculated health scores by a predetermined time interval (for example, week, month, year, and the like). The external terminal 200 used by the user U1 displays information that indicates the health score time series received from the control unit 100. As indicated in contents CT11 to CT13, the health managing system 1 executes time series displaying of a health score by using a graph in which a lateral axis thereof corresponds to time and a vertical axis thereof corresponds to a health score.

In FIG. 11, regarding to the user U1, the control unit 100 generates the content CT11 that includes a health score time series indicating change in a health score for a week. Specifically, the control unit 100 includes a health score time series that indicates, by using a bar graph, transition in a health score of the user U1 during seven days (for example, week including July 22) from Monday to Sunday. The control unit 100 transmits the content CT11 to the external terminal 200, and the external terminal 200 displays the received content CT11. The external terminal 200 displays the content CT11 including a health score time series that indicates change in a health score of the user U1 for a week.

Regarding the user U1, the control unit 100 generates the content CT12 that includes a health score time series indicating change in a health score for a month. Specifically, the control unit 100 generates the content CT12 including a health score time series that indicates, by using a bar graph, transition in a health score of the user U1 during a month (for example, July 2022). The control unit 100 transmits the content CT12 to the external terminal 200, and the external terminal 200 displays the received content CT12. The external terminal 200 displays the content CT12 including a health score time series that indicates change in a health score of the user U1 for a month.

Regarding the user U1, the control unit 100 generates the content CT13 including a health score time series that indicates change in a health score for a year. Specifically, the control unit 100 generates the content CT13 including a health score time series that indicates, by using a bar graph, transition in a health score of the user U1 during one year (for example, one year including July 2022). For each of the twelve months (January to December) of one year, the control unit 100 generates the content CT13 including a health score time series that indicates transition in average of health scores during a corresponding month. The control unit 100 transmits the content CT13 to the external terminal 200, and the external terminal 200 displays the received content CT13. The external terminal 200 displays the content CT13 including a health score time series that indicates change in a health score of the user U1 for a year.

In a case where not having acquired a health score regarding the user U1, the control unit 100 generates content CT14 including information that urges the user U1 to use the toilet 10 in order to obtain a health score. The control unit 100 transmits the content CT14 to the external terminal 200, and the external terminal 200 displays the received content CT14. The external terminal 200 displays the content CT14 including information that urges the user U1 to use the toilet 10 in order to obtain a health score.

Note that the health managing system 1 may display the content CT11 to CT13 in a switched manner. The control unit 100 transmits the contents CT11 to CT13 to the external terminal 200, and the external terminal 200 displays, in accordance with selection by the user U1, a selected content from among the contents CT11 to CT13.

### <4-4. Displaying Example of Indexes>

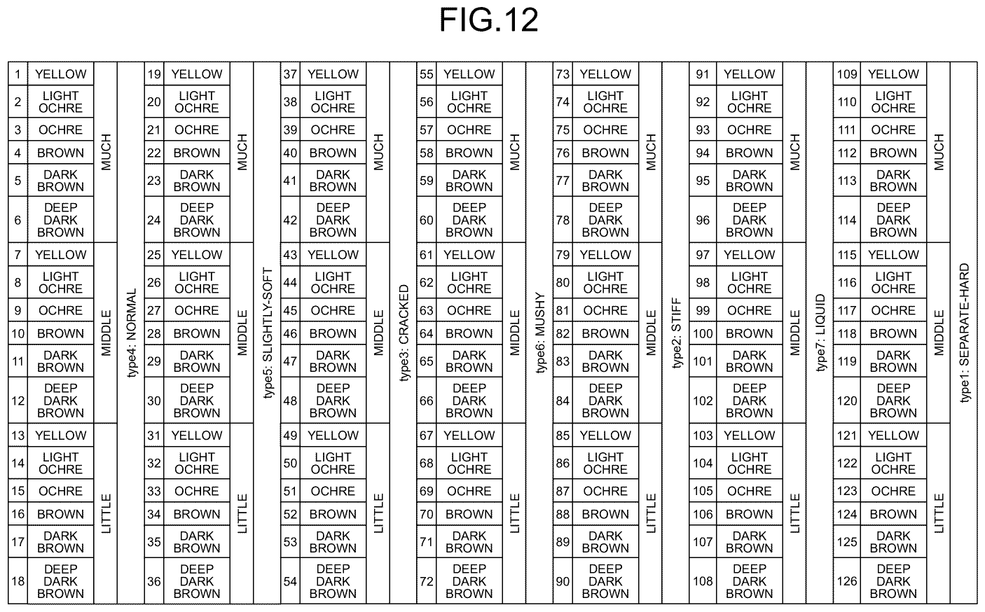

Hereinafter, displaying examples of indexes such as a stool state (namely, stool index) and a health index will be explained with reference to FIG. 12 to FIG. 16. In FIG. 12 to FIG. 16, a case will be explained as one example where a target user is the user U1, and information is displayed on the external terminal 200 such as a smartphone used by the user U1. Note that explanation of points similar to the above-mentioned is appropriately omitted.

### <4-4-1. Time-Series Displaying Example of Stool>

First, displaying examples related to a stool state will be explained with reference to FIG. 12 to FIG. 14. Herein, for example, in a case where information is displayed while targeting a long time interval such as a month and a year, amount of information to be displayed is too large in some cases. For example, in a case where a time series of an excreted stool for a year is displayed by a month unit, the number of excreted stools corresponding to a month is large (if once a day, about 30), and in a case where all of them are displayed, amount of the information becomes too large, and thus it is hard to say that the displaying is appropriate for a user. Thus, in a case where executing displaying by at least a month unit or a year unit, the health managing system 1 displays a predetermined number (may be also referred to as “number of displays”) of patterns whose frequency is large from among patterns corresponding to excreted stool properties during a predetermined time interval.

Note that the above-mentioned “pattern” indicates a combination of a type of a stool, a color of the stool, and an amount of the stool. For example, in a case where a type of a stool includes seven types, a color of a stool includes six types, and an amount of a stool includes three types; the number of combinations of a type of a stool, a color of a stool, and an amount of a stool is “126 (=7×6×3)”, and patterns corresponding to excreted stool properties are 126 patterns.

For example, in a case where the predetermined time interval is one year, and executing time series displaying about excreted stools for the year by a month unit as a calculation unit, the control unit 100 counts frequencies (namely, times) of respective patterns for each of months from January to December. For example, in a case where displaying a time series of excreted stools of the user U1 for a year of 2021 by a month unit, the control unit 100 counts frequencies of respective patterns for each of twelve months from January to December in 2021. For example, by using an excreted stool history of the user U1 stored in the storage 120, the control unit 100 calculates frequencies of respective patterns for each of twelve months from January to December in 2021. For example, in a case where the user U1 excreted, at six times during January in 2021, a stool having a pattern in which a type is “type 4 (normal)”, an amount is “middle”, and a color is “brown”; the control unit 100 calculates a frequency of a pattern during January in 2021 to be “6”, in which a type is “type 4 (normal)”, an amount is “middle”, and a color is “brown”. As described above, the control unit 100 counts frequencies of respective patterns for each of twelve months from January to December in 2021 with respect to the user U1.

Regarding the user U1, the control unit 100 decides a display number of patterns in descending order of a frequency, to be patterns (may be also referred to as “patterns to be displayed”) to be displayed for each of twelve months from January to December in 2021. For example, regarding the user U1, in a case where the number of displaying is “5”, the control unit 100 decides five patterns in descending order of a frequency, to be patterns to be displayed for each of twelve months from January to December in 2021. For example, regarding the user U1, in a case where a frequency “6” of a pattern in which a type is “type 4 (normal)”, an amount is “middle”, and a color is “brown” is the fifth most in excreted stools during January in 2021; the control unit 100 decides five patterns including patterns of a type of “type 4 (normal)”, an amount “middle”, and a color “brown” to be patterns to be displayed.

In a case where there presents a plurality of patterns to be a candidate for displaying, the health managing system 1 decides a pattern to be displayed on the basis of a predetermined reference. In a case where the number of displaying is “5” and there presents a plurality of patterns whose frequencies are the fifth most, the health managing system 1 decides a pattern to be displayed on the basis of a predetermined reference. For example, the health managing system 1 decides a pattern to be displayed on the basis of, for example, a preference order illustrated in FIG. 12, so as to display information on the decided pattern to be displayed.

FIG. 12 is a diagram illustrating one example of a preference order of a stool property. Specifically, FIG. 12 indicates a preference order list obtained by associating each combination (namely, pattern) of a type of a stool, a color of the stool, and an amount of the stool with a corresponding place. In the preference order list illustrated in FIG. 12, a pattern of the first place (namely, highest rank) indicates a combination in which a type is “type 4 (normal)”, an amount is “much”, and a color is “yellow”. In the preference order list illustrated in FIG. 12, a pattern of the 63rd place indicates a combination in which a type is “type 6 (mushy)”, an amount is “middle”, and a color is “ochre”. In the preference order list illustrated in FIG. 12, a pattern of the 126th place (namely, lowest rank) indicates a combination in which a type is “type 1 (separate-hard)”, an amount is “little”, and a color is “deep dark brown”.

In the example illustrated in FIG. 12, the health managing system 1 displays patterns on the basis of a preference order for prioritizing a type, an amount, and a color in this order. For example, the control unit 100 decides patterns to be displayed by using the preference order list illustrated in FIG. 12. For example, the control unit 100 decides patterns to be displayed with reference to a preference order list stored in the storage 120. For example, in a case where there presents a plurality of patterns corresponding to places that correspond to the number of displays, the control unit 100 decides patterns to be displayed by using the preference order list.

Regarding the user U1, a case will be explained as an example in which among excreted stools during February in 2021, both of a frequency of a first pattern in which a type is “type 3 (cracked)”, an amount is “little”, and a color is “brown”; and a frequency “6” of a second pattern in which a type is “type 4 (normal)”, an amount is “much”, and a color is “brown” are “6”, and are the fifth most. In this case, in a case where the number of displaying is “5”, a place of the first pattern is “52nd” place and a place of the second pattern is “4th” place, and thus the control unit 100 decides the second pattern to be a pattern to be displayed. Thus, regarding the user U1, among excreted stools during February in 2021, the control unit 100 decides five patterns including the second pattern in which a type is “type 4 (normal)”, an amount is “much”, and a color is “brown” to be patterns to be displayed. In other words, regarding the user U1, among excreted stools during February in 2021, the control unit 100 decides the first pattern in which a type is “type 3 (cracked)”, an amount is “little”, and a color is “brown” not to be a pattern to be displayed.

In a case where executing displaying at a time interval of a day, the health managing system 1 displays all patterns of a stool of a user on a target day (for one day). In a case where executing displaying at a time interval of a day, the control unit 100 decides all patterns of a stool of a user on a target day (for one day) to be patterns to be displayed. Note that in a case where executing displaying at a time interval of a week, the health managing system 1 may display all patterns of stools of a user during a target week (namely, one week), or may display a display number of patterns in descending order of a frequency from among patterns of stools of a user during a target week (namely, one week). In other words, in a case where executing displaying at a time interval of a week, the health managing system 1 may execute displaying similarly to a case where executing displaying at a time interval of a day, or may execute displaying similarly to a case where executing displaying at a time interval of a month or a year.

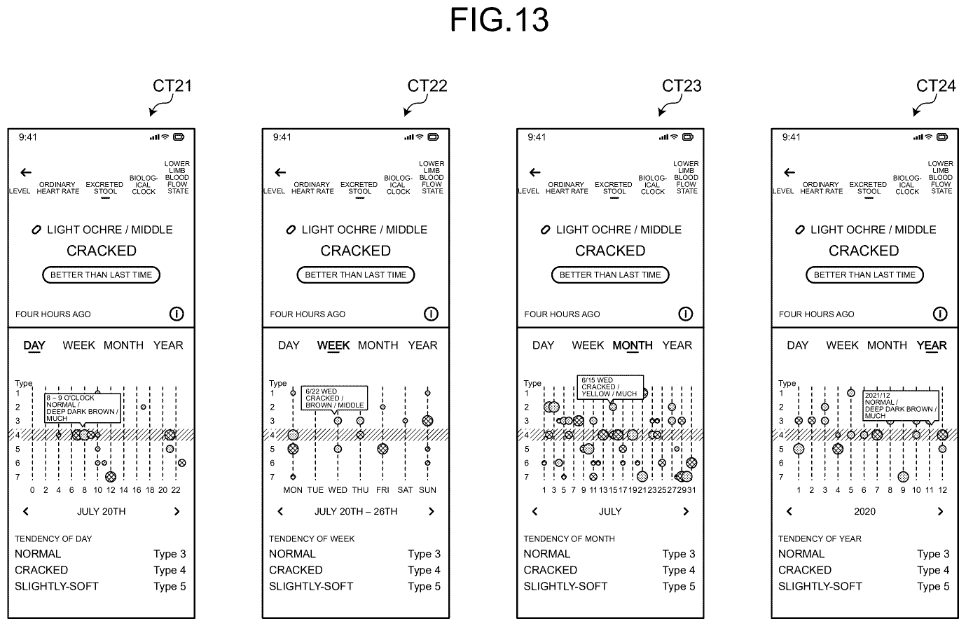

The control unit 100 generates contents CT21 to CT24 and the like illustrated in FIG. 13 while targeting the decided patterns to be displayed. FIG. 13 is a diagram illustrating one example of time series displaying of stools. Note that the contents CT21 to CT24 merely indicate images in a display mode, and thus are not intended to limit the number of displayed patterns and the like. Moreover, in a case where the contents CT21 to CT24 are explained without differentiating therebetween, they may be referred to as “contents CT20”. The control unit 100 transmits the contents CT20 to the external terminal 200, and the external terminal 200 displays the contents CT20.

As illustrated in each of the contents CT20, the health managing system 1 executes time series displaying of a stool by using a graph in which a lateral axis thereof corresponds to time and a vertical axis thereof corresponds to a pattern of a stool. Each of round-shaped points illustrated in the content CT20 corresponds to a corresponding pattern of a stool, a position of a round-shaped point in the up-and-down direction indicates a type of a stool, a color of a round-shaped point indicates a color of a stool, and a size of a round-shaped point indicates an amount of a stool. For example, a round-shaped point located in a position of 4 o'clock on a lateral axis direction in the content CT21 indicates that a stool at excretion in a time zone corresponding to 4 o'clock on July 20th corresponds to a pattern in which a type is “type 4 (normal)”, an amount is “little”, and a color is “ochre”. As described above, regarding a stool state (namely, stool index), the health managing system 1 indicates a color of a stool by using a color of a plotted point, an amount by using a size thereof, and a type of a stool by using a position on a vertical axial direction. Thus, the health managing system 1 is capable of simultaneously displaying three elements (namely, information) on a two-dimensional graph. In FIG. 13, a color of a stool is indicated by using depth of hatching, and a color is deeper as hatching is deeper. The content CT20 includes information (namely, higher pattern information) that indicates top three of the number of patterns of stools at a corresponding time interval from among a day, a week, a month, and a year. For example, in the content CT20 illustrated in FIG. 13, higher pattern information is arranged in a lower part of information indicating an excreted stool time series. Note that in calculation of higher pattern information, an arbitrary pattern may be targeted, for example, a combination (namely, pattern) of a type, an amount, and a color of a stool may be targeted, or a combination (namely, pattern) of a type and a color of a stool may be targeted.

For example, regarding the user U1, the control unit 100 causes the external terminal 200 used by the user U1 to display the content CT20 indicating a pattern of a stool that is calculated by a predetermined calculation unit during a predetermined target time interval (for example, day, week, month, year, and the like). Regarding the user U1, the control unit 100 transmits, to the external terminal 200 used by the user U1, information indicating an excreted stool time series obtained by calculating past-acquired excreted stool information at a predetermined target time interval (for example, day, week, month, year, and the like). The external terminal 200 used by the user U1 displays information indicating the excreted stool time series received from the control unit 100.

In FIG. 13, regarding the user U1, the control unit 100 generates the content CT21 that includes an excreted stool time series indicating a pattern of a stool on a day. Specifically, the control unit 100 generates the content CT21 including an excreted stool time series that indicates patterns to be displayed of the user U1 on a day (for example, July 20th) by using a graph (for example, bubble chart and the like) obtained by plotting round-shaped points thereon. The control unit 100 transmits the content CT21 to the external terminal 200, and the external terminal 200 displays the received content CT21. The external terminal 200 displays the content CT21 including an excreted stool time series that indicates a pattern of a stool of the user U1 on a day. The external terminal 200 displays the content CT21 including higher pattern information that indicates top three (in other words, first place, second place, and third place) of the number (namely, frequency) of patterns of the user U1 on a day.

Regarding the user U1, the control unit 100 generates the content CT22 including an excreted stool time series that indicates a pattern of a stool for a week.

Specifically, the control unit 100 generates the content CT22 including an excreted stool time series that indicates patterns to be displayed of the user U1 during seven days (for example, week including July 20th) from Monday to Sunday by using a graph obtained by plotting round-shaped points thereon. The control unit 100 transmits the content CT22 to the external terminal 200, and the external terminal 200 displays the received content CT22. The external terminal 200 displays the content CT22 including an excreted stool time series that indicates a pattern of a stool of the user U1 for a week. The external terminal 200 displays the content CT22 including higher pattern information that indicates top three of a frequency from among patterns of stools of the user U1 for a week.

Regarding the user U1, the control unit 100 generates the content CT23 including an excreted stool time series that indicates a pattern of a stool for a month. Specifically, the control unit 100 generates the content CT23 including an excreted stool time series that indicates patterns to be displayed of the user U1 during a month (for example, July 2022) by using a graph obtained by plotting round-shaped points thereon. The control unit 100 transmits the content CT23 to the external terminal 200, and the external terminal 200 displays the received content CT23. The external terminal 200 displays the content CT23 including an excreted stool time series that indicates a pattern of a stool of the user U1 for a month. The external terminal 200 displays the content CT23 including higher pattern information that indicates top three of a frequency from among patterns of stools of the user U1 for a month.

Regarding the user U1, the control unit 100 generates the content CT24 including an excreted stool time series that indicates a pattern of a stool for a year. Specifically, the control unit 100 generates the content CT24 including an excreted stool time series that indicates patterns to be displayed of the user U1 during a year (for example, one year including July 2022) by using a graph obtained by plotting round-shaped points thereon. Regarding each of twelve months (January to December) of one year, the control unit 100 generates the content CT24 that includes an excreted stool time series of patterns of stools during the corresponding month. The control unit 100 transmits the content CT24 to the external terminal 200, and the external terminal 200 displays the received content CT24. The external terminal 200 displays the content CT24 including an excreted stool time series that indicates a pattern of a stool of the user U1 for a year. The external terminal 200 displays the content CT24 including higher pattern information that indicates top three of a frequency from among patterns of stools of the user U1 for a year.

In a case where the number of excreted stools during a calculation unit does not reach the number of displaying even when the number of displaying during a calculation unit such as a month and a year is limited, the health managing system 1 displays patterns of all excreted stools during the calculation unit. For example, in a case where a calculation unit is a month and the number of excretions of stools during February is “3” even when a time interval is a year and the number of displaying is limited to “5”, all of the three patterns become patterns to be displayed regarding February, and the health managing system 1 displays them.

As described above, the health managing system 1 displays the contents CT21 to CT24 regarding a stool state (namely, stool index). For example, in a case where the user U1 selects the index IX6 in the content CT1, the health managing system 1 displays the content CT21. The health managing system 1 may display the contents CT21 to CT24 in a switched manner. The control unit 100 transmits the contents CT21 to CT24 to the external terminal 200, and in accordance with selection of the user U1, the external terminal 200 displays the selected content CT20 from among the contents CT21 to CT24. For example, in a case where the user U1 selects a region of the content CT20 where “week” is described, the external terminal 200 displays the content CT22.

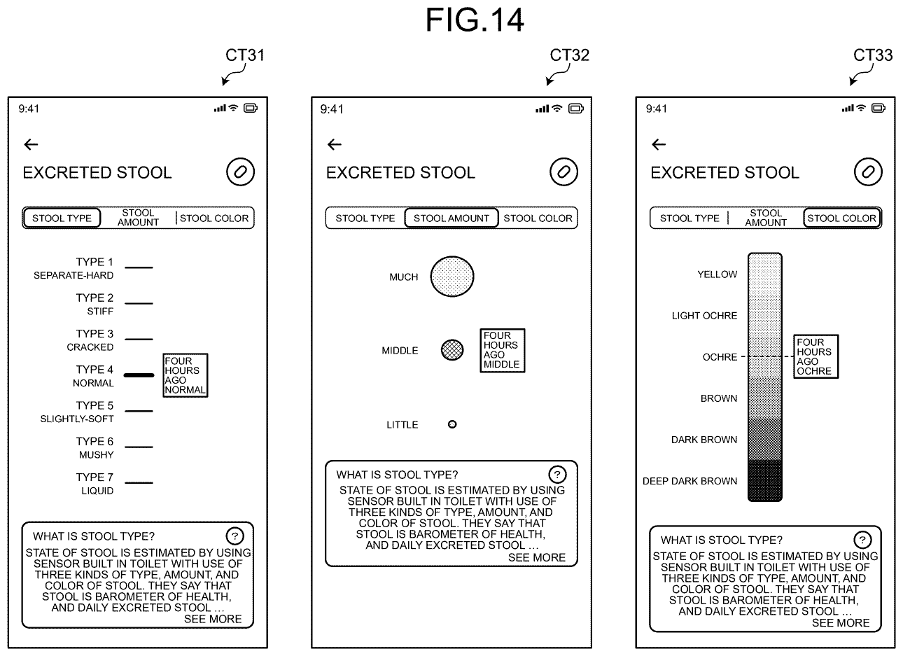

The health managing system 1 may display explanation of a stool property. The above-mentioned point will be explained with reference to FIG. 14. FIG. 14 is a diagram illustrating one example of an explanation displaying of a stool property. Moreover, in a case where contents CT31 to CT33 will be explained without differentiating therebetween, they may be referred to as “contents CT30”. The control unit 100 transmits the contents CT30 to the external terminal 200, and the external terminal 200 displays the contents CT30.

The content CT31 illustrated in FIG. 14 indicates a displaying example of information related to a type of a stool. The content CT31 includes information that indicates a type of the last acquired stool. The content CT31 includes character information that is an explanation indicating what kind of meaning information of a type of a stool has. For example, the content CT31 includes information indicating that a type of a stool corresponding to the last excreted stool excreted by the user U1 four hours ago is a type 4 (normal).

The content CT32 illustrated in FIG. 14 indicates a displaying example of information related to an amount of a stool. The content CT32 includes information indicating an amount of the last acquired stool. The content CT32 includes character information that is an explanation indicating what kind of meaning information of an amount of a stool has. For example, the content CT32 includes information indicating that an amount of a stool corresponding to the last excreted stool excreted by the user U1 four hours ago is middle.

The content CT33 illustrated in FIG. 14 indicates a displaying example of information related to a color of a stool. The content CT33 includes information indicating a color of the last acquired stool. The content CT33 includes character information that is an explanation indicating what kind of meaning information of a color of a stool has. For example, the content CT33 includes information indicating that a color of a stool corresponding to the last excreted stool excreted by the user U1 four hours ago is ochre.

As described above, the health managing system 1 displays the contents CT31 to CT33 about explanation of stool states (namely, stool indexes). For example, in a case where the user U1 selects an icon (for example, mark obtained by enclosing “i” by using circle) for displaying information in the content CT20, the health managing system 1 displays the content CT31. The health managing system 1 may display the contents CT31 to CT33 in a switched manner. The control unit 100 transmits the contents CT31 to CT33 to the external terminal 200, and in accordance with selection by the user U1, the external terminal 200 displays the selected content CT30 from among the contents CT31 to CT33. For example, in a case where the user U1 selects a region of the content CT30 where “color of stool” is described, the external terminal 200 displays the content CT33.

### <4-4-2. Time-Series Displaying Example of Health Index>

The health managing system 1 executes time series displaying on a health index similarly to a stool state.

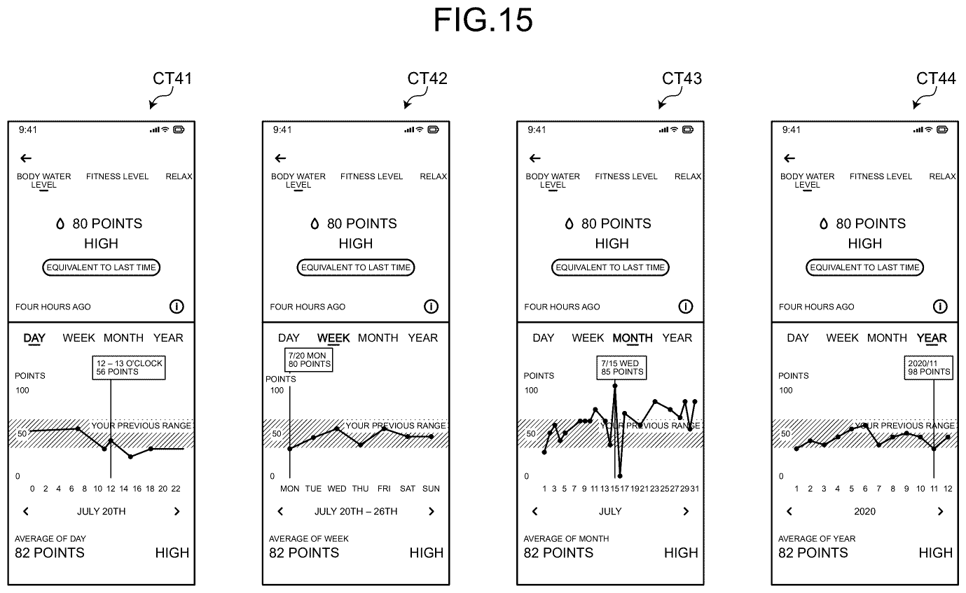

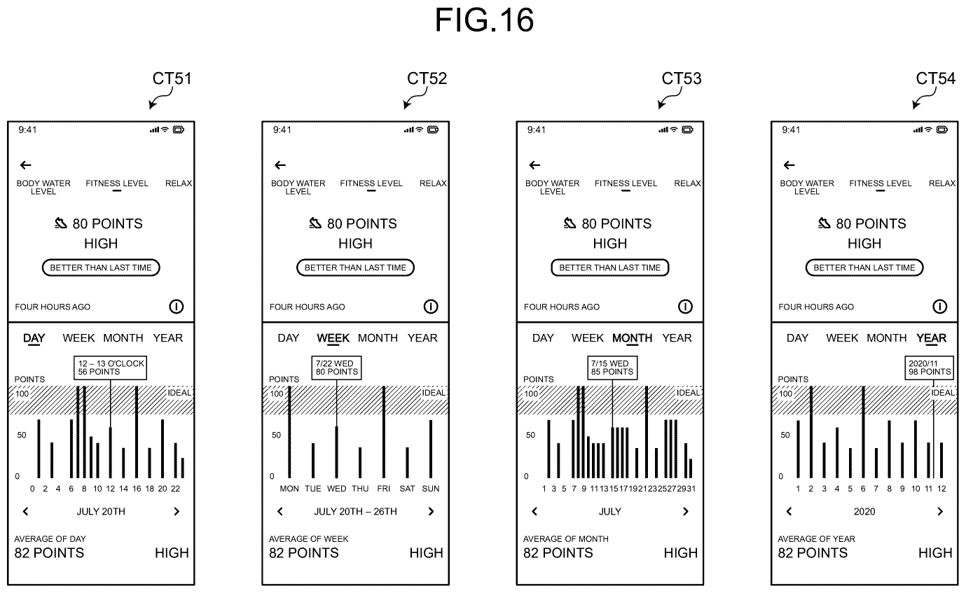

For example, the health managing system 1 acquires health index information based on biological information on a user, calculates a health index score obtained by quantifying the health index information, and displays the calculated health index score as time series data. The health managing system 1 displays an average value for each predetermined time interval as a corresponding tendency. As described above, the health managing system 1 sets the average value as a display target regarding a health index to be capable of clearly informing a user of a tendency of a health index. Examples of time series displaying of health indexes will be explained with reference to FIG. 15 and FIG. 16. FIG. 15 and FIG. 16 are diagrams illustrating examples of time series displaying of health indexes.

With reference to FIG. 15, a case will be explained as one example in which time series displaying is executed on information related to a health index by using a line graph. FIG. 15 illustrates one example of time series displaying of a health index using a line graph. In FIG. 15, a body water level will be explained to be one example of a health index.

For example, in a case where a predetermined time interval is set to a year, and time series displaying is executed on body water levels during a year at a calculation unit of each month; the control unit 100 calculates an average value of a body water level for each of the months of January to December. For example, in a case where displaying a time series of body water levels of the user U1 during a year of 2021 for each one month, the control unit 100 calculates an average value of a body water level for each of twelve months of January to December in 2021. For example, the control unit 100 calculates an average value of a body water level for each of twelve months of January to December in 2021 by using a body water level history of the user U1 stored in the storage 120.

The control unit 100 generates contents CT41 to CT44 and the like illustrated in FIG. 15 by using average values of body water levels for each calculated calculation unit. In a case where the contents CT41 to CT44 are explained without differentiating therebetween, they may be referred to as “contents CT40”. The control unit 100 transmits the contents CT40 to the external terminal 200, and the external terminal 200 displays the contents CT40.

For example, regarding the user U1, the control unit 100 causes the external terminal 200 used by the user U1 to display the content CT40 indicating a body water level that is calculated by a predetermined calculation unit during a predetermined target time interval (for example, day, week, month, year, and the like). Regarding the user U1, the control unit 100 transmits, to the external terminal 200 used by the user U1, information indicating a body water level time series obtained by calculating the past acquired body water levels at a predetermined target time interval (for example, day, week, month, year, and the like). The external terminal 200 used by the user U1 displays information that indicates the body water level time series received from the control unit 100. The content CT40 includes information (namely, average state information) that indicates a state based on average during a time interval corresponding one of a day, a week, a month, and a year. For example, in the content CT40 illustrated in FIG. 15, average state information on a body water level is arranged in a lower part of information indicating a body water level time series.

In FIG. 15, regarding the user U1, the control unit 100 generates the content CT41 including a body water level time series that indicates transition in a body water level on a day. Specifically, the control unit 100 generates the content CT41 including a body water level time series that indicates an average value of a body water level of the user U1 on a day (for example, July 20th) by using a line graph. The control unit 100 transmits the content CT41 to the external terminal 200, and the external terminal 200 displays the received content CT41. The external terminal 200 displays the content CT41 including a body water level time series that indicates transition in a body water level of the user U1 on a day. The external terminal 200 displays the content CT41 that includes average state information on a body water level based on average of a body water level of the user U1 on a day.

Regarding the user U1, the control unit 100 generates the content CT42 including a body water level time series that indicates transition in a body water level for a week. Specifically, the control unit 100 generates the content CT42 including a body water level time series that indicates an average value of a body water level of the user U1 during seven days (for example, week including July 20th) from Monday to Sunday by using a line graph.

The control unit 100 transmits the content CT42 to the external terminal 200, and the external terminal 200 displays the received content CT42. The external terminal 200 displays the content CT42 including a body water level time series that indicates transition in a body water level of the user U1 for a week. The external terminal 200 displays the content CT42 that includes average state information on a body water level based on average of a body water level of the user U1 for a week.

Regarding the user U1, the control unit 100 generates the content CT43 including a body water level time series that indicates transition in a body water level for a month. Specifically, the control unit 100 generates the content CT43 including a body water level time series that indicates an average value of a body water level of the user U1 during a month (for example, July 2022) by using a line graph. The control unit 100 transmits the content CT43 to the external terminal 200, and the external terminal 200 displays the received content CT43. The external terminal 200 displays the content CT43 including a body water level time series that indicates transition in a body water level of the user U1 for a month. The external terminal 200 displays the content CT43 that includes average state information on a body water level based on average of a body water level of the user U1 for a month.

Regarding the user U1, the control unit 100 generates the content CT44 including a body water level time series that indicates transition in a body water level for a year. Specifically, the control unit 100 generates the content CT44 including a body water level time series that indicates an average value of a body water level of the user U1 for a year (for example, one year including July 2022) by using a line graph. Regarding each of twelve months (January to December) of one year, the control unit 100 generates the content CT44 that includes a body water level time series indicating transition in a body water level during the corresponding month. The control unit 100 transmits the content CT44 to the external terminal 200, and the external terminal 200 displays the received content CT44. The external terminal 200 displays the content CT44 including a body water level time series that indicates transition in a body water level of the user U1 for a year. The external terminal 200 displays the content CT44 that includes average state information on a body water level based on average of a body water level of the user U1 for a year.

As described above, the health managing system 1 displays the contents CT41 to CT44 regarding a body water level (namely, health index). For example, in a case where the user U1 selects the index IX1 in the content CT1, the health managing system 1 displays the content CT41. The health managing system 1 may display the contents CT41 to CT44 in a switched manner. The control unit 100 transmits the contents CT41 to CT44 to the external terminal 200, and in accordance with selection of the user U1, the external terminal 200 displays the selected content CT40 from among the contents CT41 to CT44. For example, in a case where the user U1 selects a region of the content CT40 where “week” is described, the external terminal 200 displays the content CT42.

Next, with reference to FIG. 16, a case will be explained as one example in which time series displaying is executed on information related to a health index by using a bar graph. FIG. 16 illustrates one example of time series displaying of a health index using a bar graph. Note that in FIG. 16, a fitness level will be explained as one example of a health index.

For example, in a case where a predetermined time interval is set to a year and time series displaying is executed on fitness levels during a year at a calculation unit of each month; the control unit 100 calculates an average value of a fitness level for each of the months of January to December. For example, in a case where displaying a time series of fitness levels of the user U1 during a year of 2021 for each one month, the control unit 100 calculates an average value of a body water level for each of twelve months of January to December in 2021. For example, the control unit 100 calculates an average value of a fitness level for each of twelve months of January to December in 2021 by using a fitness level history of the user U1 stored in the storage 120.

The control unit 100 generates contents CT51 to CT54 and the like illustrated in FIG. 16 by using average values of fitness levels for each calculated calculation unit. In a case where the contents CT51 to CT54 will be explained without differentiating therebetween, they may be referred to as “contents CT50”. The control unit 100 transmits the contents CT50 to the external terminal 200, and the external terminal 200 displays the contents CT50.

For example, regarding the user U1, the control unit 100 causes the external terminal 200 used by the user U1 to display the content CT50 indicating a fitness level that is calculated by a predetermined calculation unit during a predetermined target time interval (for example, day, week, month, year, and the like). Regarding the user U1, the control unit 100 transmits, to the external terminal 200 used by the user U1, information indicating a fitness level time series obtained by calculating the past acquired fitness levels at a predetermined target time interval (for example, day, week, month, year, and the like). The external terminal 200 used by the user U1 displays information that indicates the fitness level time series received from the control unit 100. The content CT50 includes information (namely, average state information) that indicates a state based on average during a time interval corresponding one of a day, a week, a month, and a year. For example, in the content CT50 illustrated in FIG. 15, average state information on a fitness level is arranged in a lower part of information indicating a fitness level time series.

In FIG. 16, regarding the user U1, the control unit 100 generates the content CT51 including a fitness level time series that indicates transition in a fitness level on a day. Specifically, the control unit 100 generates the content CT51 including a fitness level time series that indicates an average value of a fitness level of the user U1 on a day (for example, July 20th) by using a bar graph. The control unit 100 transmits the content CT51 to the external terminal 200, and the external terminal 200 displays the received content CT51. The external terminal 200 displays the content CT51 including a fitness level time series that indicates transition in a fitness level of the user U1 on a day. The external terminal 200 displays the content CT51 that includes average state information on a fitness level based on average of a fitness level of the user U1 on a day.

Regarding the user U1, the control unit 100 generates the content CT52 including a fitness level time series that indicates transition in a fitness level for a week. Specifically, the control unit 100 generates the content CT52 including a fitness level time series that indicates an average value of a fitness level of the user U1 during seven days (for example, week including July 20th) from Monday to Sunday by using a bar graph. The control unit 100 transmits the content CT52 to the external terminal 200, and the external terminal 200 displays the received content CT52. The external terminal 200 displays the content CT52 including a fitness level time series that indicates transition in a fitness level of the user U1 for a week. The external terminal 200 displays the content CT52 that includes average state information on a fitness level based on average of a fitness level of the user U1 for a week.

Regarding the user U1, the control unit 100 generates the content CT53 including a fitness level time series that indicates transition in a fitness level for a month. Specifically, the control unit 100 generates the content CT53 including a fitness level time series that indicates an average value of a fitness level of the user U1 during a month (for example, July 2022) by using a bar graph. The control unit 100 transmits the content CT53 to the external terminal 200, and the external terminal 200 displays the received content CT53. The external terminal 200 displays the content CT53 including a fitness level time series that indicates transition in a fitness level of the user U1 for a month. The external terminal 200 displays the content CT53 that includes average state information on a fitness level based on average of a fitness level of the user U1 for a month.

Regarding the user U1, the control unit 100 generates the content CT54 including a fitness level time series that indicates transition in a fitness level for a year. Specifically, the control unit 100 generates the content CT54 including a fitness level time series that indicates an average value of a fitness level of the user U1 during a year (for example, one year including July 2022) by using a bar graph. Regarding each of twelve months (January to December) of one year, the control unit 100 generates the content CT54 that includes a fitness level time series indicating transition in a fitness level during the corresponding month. The control unit 100 transmits the content CT54 to the external terminal 200, and the external terminal 200 displays the received content CT54. The external terminal 200 displays the content CT54 including a fitness level time series that indicates transition in a fitness level time series of the user U1 for a year. The external terminal 200 displays the content CT54 that includes average state information on a fitness level based on average of a fitness level of the user U1 for a year.

As described above, the health managing system 1 displays the contents CT51 to CT54 regarding a fitness level (namely, health index). For example, in a case where the user U1 selects the index IX2 in the content CT1, the health managing system 1 displays the content CT51. The health managing system 1 may display the contents CT51 to CT54 in a switched manner. The control unit 100 transmits the contents CT51 to CT54 to the external terminal 200, and in accordance with selection of the user U1, the external terminal 200 displays the content CT50 from among the contents CT51 to CT54. For example, in a case where the user U1 selects a region of the content CT50 where “week” is described, the external terminal 200 displays the content CT52.

In the above-mentioned example, displaying examples are explained about two health indexes of a body water level and a fitness level; however, the health managing system 1 is capable of similarly executing time series displaying on other health indexes such as a heart rate, a stress state, a blood circulation state, a metabolic level, a blood vessel age, and a biological clock. The above-mentioned display is merely one example, and the health managing system 1 may display indexes such as a stool state (namely, stool index) and a health index in various display modes.

The above-mentioned embodiments and modifications may be appropriately combined within a consistent range of processing details.

The above-mentioned embodiments and modifications are described by using an example in which information on indexes is automatically acquired; however, a user him/herself may manually input the information on indexes. For example, in a case where a user him/herself manually input information related to a stool, the user visually and/or olfactorily recognizes him/her stool after excretion, and further operates the external terminal 200 so as to input information related to the stool.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

The above-mentioned embodiments and modifications may be practiced by the following configuration; however, not limited thereto.

(1)

A health managing system including:
an excreted stool information acquiring unit that acquires excreted stool information corresponding to an excretion of a stool of a user;
an excreted stool state determining unit that determines an excreted stool property of an excreted stool corresponding to the excreted stool information based on the excreted stool information acquired by the excreted stool information acquiring unit;
a display to be browsed by the user; and
a display processing unit that executes a process for causing the display to display, as time series data, the excreted stool property that is determined by the excreted stool state determining unit, wherein
the display processing unit is further configured to:
   be capable of switching displaying for each predetermined time interval; and
   from among excreted stool property patterns during the predetermined time interval, execute a process for causing the display to display a predetermined number of ones whose frequency is large.
      (2)

The health managing system according to (1), wherein
the display processing unit is further configured to:
   execute a process for switching the displaying on the display from among a day unit, a week unit, a month unit, and a year unit.
      (3)

The health managing system according to (1) or (2), wherein
the display processing unit is further configured to:
   in a case where executing displaying by at least a month unit or a year unit, execute a process for causing the display to display a predetermined number of patterns whose frequency is large from among patterns corresponding to excreted stool properties during the predetermined time interval.
      (4)

The health managing system according to any one of (1) to (3), wherein
the display processing unit is further configured to:
   in a case where executing displaying by at least a day unit, display all excreted stool properties of the user determined by the excreted stool state determining unit.
      (5)

The health managing system according to any one of (1) to (4), wherein
the excreted stool state determining unit is further configured to:
   determine the excreted stool property including a type, an amount, and a color of an excreted stool, and
the display processing unit is further configured to:
   execute a process for causing the display to execute displaying based on a pattern corresponding to a combination of the type, the amount, and the color.
      (6)

The health managing system according to (5), wherein
the display processing unit is further configured to:
   execute a process for causing the display to display the pattern based on a preference order of the type, the amount, and the color in this order.
      (7)

A health managing method comprising:
acquiring excreted stool information corresponding to an excretion of a stool of a user;
determining an excreted stool property of an excreted stool corresponding to the excreted stool information based on the excreted stool information acquired in the acquiring; and
executing a process for causing a display to be browsed by the user to display, as time series data, the excreted stool property that is determined in the determining, wherein
the executing includes:
   being capable of switching displaying for each predetermined time interval; and
   from among excreted stool property patterns during the predetermined time interval, executing a process for causing the display to display a predetermined number of ones whose frequency is large.
      (8)

A health managing system comprising:
an excreted stool information acquiring unit that acquires excreted stool information corresponding to an excretion of a stool of a user;
an excreted stool state determining unit that determines an excreted stool property of an excreted stool corresponding to the excreted stool information based on the excreted stool information acquired by the excreted stool information acquiring unit;
a display to be browsed by the user; and
a display processing unit that executes a process for causing the display to display, as time series data, the excreted stool property that is determined by the excreted stool state determining unit, wherein
the excreted stool state determining unit is further configured to:
   determine a type of a stool, an amount of the stool, and a color of the stool, and
the display processing unit is further configured to execute a process including:
   executing time series displaying on stool by using a graph in which a lateral axis corresponds to time and a vertical axis corresponds to a pattern of a stool such that a color of each point in the graph indicates a color of a stool, and a size of the corresponding point indicates an amount of the stool.

### REFERENCE SIGNS LIST

1 Health managing system
4 Closet bowl
4b Upper surface
10 Toilet (Toilet system)
12 Main body part
14 Toilet lid
20 Toilet seat
20a Opening
21 Seating surface
25 Bottom surface
40 Biological sensor (Biological information acquisition unit)
50 Seating sensor (Electrostatic-type sensor)
60 Excreted stool sensor
70 Timer
100 Control unit (Information processing device)
110 Acquisition unit
120 Storage
130 Health index calculating unit
140 Excreted stool information calculating unit (Excreted stool state determining unit)
150 Health state calculating unit
160 Display processing unit
161 Health state display processing unit
162 Health index display processing unit
163 Excreted stool state display processing unit
164 Measurement situation display processing unit
165 Message display processing unit
166 Highlight display processing unit
190 Communication unit
200 External terminal
CL Cloud

What is claim is:

1. A health managing system comprising:
an excreted stool information acquiring unit that acquires excreted stool information corresponding to an excretion of a stool of a user;
an excreted stool state determining unit that determines an excreted stool property of an excreted stool corresponding to the excreted stool information based on the excreted stool information acquired by the excreted stool information acquiring unit;
a display to be browsed by the user; and
a display processing unit that executes a process for causing the display to display, as time series data, the excreted stool property that is determined by the excreted stool state determining unit, wherein
the display processing unit is further configured to:
be capable of switching displaying for each predetermined time interval; and
from among excreted stool property patterns during the predetermined time interval, execute a process for causing the display to display a predetermined number of ones whose frequency is large.

2. The health managing system according to claim 1, wherein
the display processing unit is further configured to:
execute a process for switching the displaying on the display from among a day unit, a week unit, a month unit, and a year unit.

3. The health managing system according to claim 1, wherein
the display processing unit is further configured to:
in a case where executing displaying by at least a month unit or a year unit, execute a process for causing the display to display a predetermined number of patterns whose frequency is large from among patterns corresponding to excreted stool properties during the predetermined time interval.

4. The health managing system according to claim 1, wherein
the display processing unit is further configured to:
in a case where executing displaying by at least a day unit, display all excreted stool properties of the user determined by the excreted stool state determining unit.

5. The health managing system according to claim 1, wherein
the excreted stool state determining unit is further configured to:
determine the excreted stool property including a type, an amount, and a color of an excreted stool, and
the display processing unit is further configured to:
execute a process for causing the display to execute displaying based on a pattern corresponding to a combination of the type, the amount, and the color.

6. The health managing system according to claim 5, wherein
the display processing unit is further configured to:
execute a process for causing the display to display the pattern based on a preference order of the type, the amount, and the color in this order.

7. A health managing method comprising:
acquiring excreted stool information corresponding to an excretion of a stool of a user;
determining an excreted stool property of an excreted stool corresponding to the excreted stool information based on the excreted stool information acquired in the acquiring; and
executing a process for causing a display to be browsed by the user to display, as time series data, the excreted stool property that is determined in the determining, wherein
the executing includes:
being capable of switching displaying for each predetermined time interval; and
from among excreted stool property patterns during the predetermined time interval, executing a process for causing the display to display a predetermined number of ones whose frequency is large.

8. A health managing system comprising:
an excreted stool information acquiring unit that acquires excreted stool information corresponding to an excretion of a stool of a user;
an excreted stool state determining unit that determines an excreted stool property of an excreted stool corresponding to the excreted stool information based on the excreted stool information acquired by the excreted stool information acquiring unit;
a display to be browsed by the user; and
a display processing unit that executes a process for causing the display to display, as time series data, the excreted stool property that is determined by the excreted stool state determining unit, wherein
the excreted stool state determining unit is further configured to:
determine a type of a stool, an amount of the stool, and a color of the stool, and
the display processing unit is further configured to execute a process including:
executing time series displaying on stool by using a graph in which a lateral axis corresponds to time and a vertical axis corresponds to a pattern of a stool such that a color of each point in the graph indicates a color of a stool, and a size of the corresponding point indicates an amount of the stool.
